(19) United States
(12) Patent Application Publication
(10) Pub. No.: US 2024/0218311 A1
(43) Pub. Date: Jul. 4, 2024

(54) SYSTEMS AND METHODS FOR RECYCLING CELL CULTURE MEDIUM

(71) Applicant: FUTURE MEAT TECHNOLOGIES LTD., Rehovot (IL)

(72) Inventors: Yaakov NAHMIAS, Mevaseret Zion (IL); Gidon HALAF, Rehovot (IL); Zach SHIDLOVSKY, Reut (IL); Guy WISSOTSKY, Rishon LeZion (IL)

(73) Assignee: FUTURE MEAT TECHNOLOGIES LTD., Rehovot (IL)

(21) Appl. No.: 18/560,214

(22) PCT Filed: May 9, 2022
(86) PCT No.: PCT/IB2022/054287

§ 371 Date: Nov. 10, 2023

Related U.S. Application Data
(60) Provisional application No. 63/186,334, filed on May 10, 2021.

(51) Int. Cl. C12M 1/00; B01J 41/05; C12M 1/12; C12N 5/078

(52) U.S. Cl. CPC C12M 29/18; B01J 41/05; C12M 29/16; C12M 37/02; C12N 5/0634; C12N 2500/14

(57) ABSTRACT

The present disclosure provides, in part, a system for recycling a cell culture medium. Also provided is a method for recycling a cell culture medium. Such system and method can be used to produce culture meat or remove waste products from a patient's blood.

### CROSS-REFERENCE TO RELATED APPLICATION

This application claims the benefit of U.S. Provisional Application No. 63/186,334, filed May 10, 2021 the disclosure of which is hereby incorporated by reference in its entirety.

### FIELD

The present disclosure generally relates to liquid filtering and recycling. More specifically, the present disclosure relates to a system and a method for recycling a cell culture medium as well as methods of expanding cells in the medium and thereby producing cultured meat.

### BACKGROUND

There are basically two main processes for large scale biological manufacturing of cells, proteins, or vaccines: either fed-batch or perfusion. In fed-batch process, cells grow in bioreactors with volumes as large as 25,000 liters and are continuously fed with nutrients until toxins reach a threshold (usually ammonia of 5 mM) and cells reach densities of up to 30 million cells/ml. In perfusion process, a medium is continuously replaced by filtering the cell suspension through a membrane (usually a hollow fiber membrane). This allows the toxins to be washed away, while allowing the cells to reach densities of up to 270 million cells/ml with bioreactors as big as 5,000 liters. However, the perfusion process wastes multiple of vessel volumes of culture media, making production more expensive than fed-batch process (albeit with 30% less expensive factory).

U.S. Pat. No. 5,071,561 discloses a method and apparatus for removing ammonia from cell cultures by contacting an aqueous culture medium with one side of a supported-fluid membrane wherein the support is a microporous hydrophobic polymeric membrane matrix; and maintaining a strip solution in contact with the other side of said membrane. Fidel Rey et al. (Cytotechnology 6: 121-130; 1991) discloses selective removal of ammonia from animal cell culture by utilizing a zeolite packed bed.

The current world population is over 7 billion and still rapidly growing. In order to support the nutritional requirement of this growing population, an increasing amount of land is dedicated for food production. Current natural sources are insufficient to fulfill the increasing demand for animal protein. This has led to famine in some parts of the world. In other parts of the world, the problem is being addressed by large-scale production of animals in dense factory farms under harsh conditions. This large-scale production not only causes great suffering to animals, but also increases arsenic levels and drug resistance bacteria in meat products due to organoarsenic compounds and antibiotics used to increase food efficiency and control infection, thus further increases the number of diseases and worsens the consequences thereof for both animals and humans. Large-scale slaughtering is required to fulfill the current food requirements and as a consequence, it can lead to large-scale disease outbreaks such as the occurrence of porcine pestivirus, avian influenza and mad cow disease. These diseases result in loss of the meat for human consumption thus completely denying the purpose for which the animals were being bred in the first place.

In addition, the large-scale production reduces the flavor of the finished product. A preference exists among those that can afford non-battery laid eggs and non-battery produced meat. It is not only a matter of taste, but also a healthier choice thereby avoiding consumption of various feed additives such as growth hormones. Another problem associated with mass animal production is the environmental problem caused by the vast amounts of fecal matter from the animals and which the environment subsequently has to deal with. Moreover, the large amount of land currently required for the production of animals or the feed for the animals which cannot be used for alternative purposes such as growth of other crops, housing, recreation, wild nature and forests is problematic.

Chicken meat has been a major source of dietary protein since the dawn of the agricultural revolution. Production has traditionally been local, with families and later small villages growing their own grain-fed animals. However, rapid urbanization and population growth driven by the industrial revolution led to the development of intensive farming methods. Factory farms now produce close to 9 billion chickens each year in the United States, with animal growth and transportation producing 18% of current greenhouse emissions. It was recognized by the present inventor that large amount of chicken meat (e.g., over 70% in the United States) contains unsafe levels of arsenic, and antibiotic resistant bacteria. It was also recognized that transportation and animal density led to widespread fecal contamination of chicken meat leading to increased salmonella infection.

Laboratory-grown meat allows growing meat from animal cells under sterile conditions. It was found by the present inventor that it is possible to produce a sufficient amount of cells per unit mass of meat product (e.g., from about 500 to about 200×10⁶ cells per gram), without the use of animal products, such as fetal bovine serum. However, while many cell culture techniques have been developed over the past 50 years for biological research, the present inventor found that such culture techniques are incredibly wasteful, requiring a large volume of culture medium to produce a small mass oflaboratory-grown meat. For example, known techniques require a volume of about 230 liters to produce about 1 kg of meat, translating to a cost of at least $4,600 per kg due to medium costs alone.

One of the primary problems of the techniques known in the art is that, with a long time to produce, and at extremely high costs, products are of a mediocre quality that cannot and will not replace the current meat derived from livestock. For example, Just-Inc. grows extracted animal cells in media to manufacture chicken nuggets, which cost $50 per nugget to manufacture.

Culture of cells, e.g., mammalian cells or insect cells, for in vitro experiments or ex vivo culture, for administration to a human or animal is an important tool for studies and treatments of human diseases. Cell culture is widely used for the production of various biologically active products, e.g., viral vaccines, monoclonal antibodies, polypeptide growth factors, hormones, enzymes, tumor specific antigens and food products. However, many of the media or methods used to culture the cells comprise components that can have negative effects on cell growth and/or maintenance of an undifferentiated cell culture. For example, mammalian or insect cell culture media is often supplemented with blood-derived serum such as fetal calf serum (FCS) or fetal bovine serum (FBS) in order to provide growth factors, carrier proteins, attachment and spreading factors, nutrients and trace elements that promote proliferation and growth of cells in culture. However, the factors found in FCS or FBS, such as transforming growth factor (TGF) beta or retinoic acid, can promote differentiation of certain cell types (Ke et al., Am J Pathol. 137: 833-43, 1990) or initiate unintended downstream signaling in the cells that promotes unwanted cellular activity in culture (Veldhoen et al., Nat Immunol. 7(11): 1151-6, 2006).

The cost of culture medium is the primary driving factor of the cost of cultured meat production. Culture medium is composed of relatively simple basal medium that comprises carbohydrates, amino acids, vitamins and minerals and much more expensive serum replacement component including; albumin, growth factors, enzymes, attachment factors and hormones. Recent analysis by the Good Food Institute suggests that serum replacement proteins represent over 99% of culture medium costs.

There is a need for cost effective cell culture media and for improved systems or methods of effectively filtering waste materials from cell culture media and recycling the media for large scale biological manufacturing of cells, proteins, or vaccines. The present invention fulfills this long-standing need.

### SUMMARY

The present disclosure provides, in part, systems and methods for separating essential materials from waste materials in a liquid medium, rejuvenating and recycling the medium for continuous use. While the systems or methods may be used for treating a vast array of liquid formulations or compositions, the present disclosure focuses on using these systems as efficient and simple ways to separate waste components from essential components of cell culture media and recycle the media for continuous use.

Accordingly, one aspect of the present disclosure provides a system for recycling a cell culture medium. Such system comprises means for removing a cell culture medium from a bioreactor, means for filtering the cell culture medium, thereby obtaining a waste medium and a concentrate medium. The waste medium comprises at least one waste material and may be devoid of any cells and large proteins. While the concentrate medium is circulated back into the bioreactor, the waste medium is further processed to obtain a rejuvenated medium. The rejuvenated medium may be diminished or essentially devoid of at least one waste material.

To further process the waste medium, in some aspects the system comprises means for acidifying the waste medium, and means for subjecting the acidified waste medium to nanofiltration, thereby removing or reducing the concentration of at least one waste material from the waste medium and obtaining a rejuvenated medium that is essentially diminished/devoid of at least one waste material. The rejuvenated medium may be circulated back into the bioreactor, thereby recycling the cell culture medium. In some aspects the rejuvenated medium flows directly back into the system in a loop. In some aspects the rejuvenated medium may be removed from the system and stored for future use. In some aspects the rejuvenated medium may be further mixed with fresh medium or recycled medium before recirculation or future use. In some aspects the waste medium can be removed from the system and processed using a standalone system to obtain a rejuvenated medium.

In some embodiments, the cell culture medium comprises one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials. By way of non-limiting example, the cell culture medium may comprise blood cells.

In some embodiments, the waste material(s) interferes with desired growth and/or desired differentiation of the cells, which include, but are not limited to, ammonia, lactate, toxins, sodium salts, alanine, glutamic acid, aspartic acid, ammonium, reactive oxygen, and nitrogen species. In some embodiments, the waste materials have a molecular weight of no greater than 60 kDa. By way of non-limiting example, the at least one waste material may comprise ammonia, ammonium, and/or lactate.

In some embodiments, the cell culture medium contains tissues cultured for antibody production, or cultured meat production. While the waste materials are removed from the cell culture medium, any produced antibodies and produced cultured meat are retained in the cell culture medium. In some aspects the system comprises a filtering means, or a centrifugation means or both. In some aspects the filtering means may comprise a alternating tangential flow (ATF) filtration system. In some aspects the ATF may comprise microfiltration means. In some aspects the ATF may comprise ultrafiltration means. In some embodiments, the filtering means comprises at least one hollow fiber. In some embodiments, the porosity profile of the hollow fiber walls is configured to provide an average pore size and pore density that only permits passage of molecules that are smaller than 60 kDa. In some embodiments, the pore density is at least 10% of the wall surface of each hollow fiber.

In some embodiments, the filter means comprises density centrifugation or other forms of continuous centrifugation, thereby producing waste medium essentially devoid of cells and large proteins. In some embodiments, the filtering means may include ultrasound cell retention or rotating drum and crossflow filtration.

In some embodiments, the concentrate medium comprises cells and essential materials for cell growth and/or differentiation and is circulated back into the bioreactor after going through the filtering means.

While the concentrate medium is circulated back into the bioreactor, the waste medium goes through further processing. In some aspects the waste medium may flow directly into an acidifying means. In some aspects the waste medium first flows through an ultrafiltration means and then further channeled to an acidifying means.

In some embodiments, the means for acidifying the waste medium comprises subjecting the waste medium to a cation exchange column and/or adding an acid to the waste medium. In some embodiments, the cation exchange column comprises at least one cation resin. By way of non-limiting example, the cation exchange column may comprise AmberLite FPC88.

In some embodiments, an acid may be added to acidify the waste medium. By way of non-limiting example, the acid may be HCl, sulfuric acid, nitric acid, phosphoric acid, carbonic acid, citric acid, or acetic acid. In some embodiments, the acidified waste medium has a pH value of less than 4. By way of non-limiting example, the acidified waste medium has a pH value of about 2.

The acidified waste medium further goes through nanofiltration. In some embodiments, the nanofiltration is also performed as a diafiltration mode which involves pre-diluting the acidified waste medium with deionized water before the nanofiltration. In some embodiments, the nanofiltration has a molecular weight cutoff of from about 150 to about 300 Da.

In some embodiments, the waste materials are recovered from the acidified waste medium post nanofiltration, which include, but are not limited to, ammonia, ammonium, and lactate. In some embodiments, these components are further isolated and recovered individually. The recovered individual components may possess commercial values that can be sold as individual products.

In some embodiments, the rejuvenated medium comprises glucose and fatty acids having a molecular weight greater than 150 Da, and is further processed by means of neutralizing the pH thereof. In some embodiments, the neutralizing means comprises subjecting the rejuvenated medium to an anion exchange column. In some embodiments, the anion exchange column comprises at least one anion resin. By way of non-limiting example, the anion exchange column may comprise FPA55.

In some embodiments, a base may be added to neutralize the acidity of the rejuvenated medium. By way of non-limiting example, the base may be NaOH, sodium bicarbonate, potassium hydroxide, magnesium hydroxide, or calcium hydroxide. In some embodiments, the pH of the rejuvenated medium is adjusted to pH>6. By way of non-limiting example, the rejuvenated medium has a pH of about 7.

In some embodiments, the osmolarity of the rejuvenated medium is adjusted to be less than 360 milliosmoles per kilogram (mOsm/kg) of water. By way of non-limiting example, the rejuvenated medium has an osmolarity of about 280 mOsm/kg.

For any of the systems described above and herein, biomass is expanded in the cell culture medium to produce cultured meat.

Another aspect of the present disclosure provides a method for recycling a cell culture medium. Such method comprises removing a cell culture medium from a bioreactor; filtering the cell culture medium, thereby obtaining a waste medium and a concentrate medium; acidifying the waste medium; and subjecting the acidified waste medium to nanofiltration, thereby removing the at least one waste material from the waste medium and obtaining a rejuvenated medium that is diminished in at least one waste material for recycling. In some embodiments, the rejuvenated medium is essentially devoid of at least one waste material for recycling. In this method, upon filtration, the waste medium comprises at least one waste material and is essentially devoid of cells and large proteins, and the concentrate medium is diminished/devoid in at least one waste material. While the concentrate medium is circulated back into the bioreactor, the waste medium is further processed.

In some embodiments, the cell culture medium comprises one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials. By way of non-limiting example, the cell culture medium may comprise blood cells.

In some embodiments, the waste material(s) interferes with desired growth and/or desired differentiation of the cells, which include, but are not limited to, ammonia, lactate, toxins, sodium salts, alanine, glutamic acid, aspartic acid, ammonium, reactive oxygen and nitrogen species. In some embodiments, the waste materials have a molecular weight of no greater than 60 kDa. By way of non-limiting example, the at least one waste material may comprise ammonia, ammonium, and/or lactate.

In some embodiments, the cell culture medium contains tissues cultured for antibody production, or cultured meat production. While the waste materials are removed from the cell culture medium, any produced antibodies and produced cultured meat are retained in the cell culture medium.

In some embodiments, the cell culture medium that exit the bioreactor flows through at least one hollow fiber for filtering. In some embodiments, the porosity profile of the hollow fiber walls is configured to provide an average pore size and pore density that only permits passage of molecules that are smaller than 60 kDa. In some embodiments, the pore density is at least 10% of the wall surface of each hollow fiber.

In some embodiments, the concentrate medium comprises cells and essential materials for cell growth and/or differentiation and is circulated back into the bioreactor for continuous use.

While the concentrate medium is circulated back into the bioreactor, the waste medium goes through further processing. In some embodiments, the waste medium is subjected to a cation exchange column and/or addition of an acid. In some embodiments, the cation exchange column comprises at least one cation resin. By way of non-limiting example, the cation exchange column may comprise AmberLite FPC88.

In some embodiments, an acid may be added to acidify the waste medium. In some embodiments, the acidified waste medium has a pH value of less than 4. By way of non-limiting example, the acidified waste medium has a pH value of about 2.

The acidified waste medium is further subjected to nanofiltration. In some embodiments, the nanofiltration is also performed as a diafiltration mode which involves pre-diluting the acidified waste medium with deionized water before the nanofiltration. In some embodiments, the nanofiltration has a molecular weight cutoff of from about 150 to about 300 Da.

In some embodiments, the methods described above and herein may further comprise recovering the waste materials from the acidified waste medium post nanofiltration; isolating the components of the waste materials; and recovering the individual component. In some embodiments, the waste materials comprise ammonia, ammonium, and/or lactate, and the recovered individual components may possess commercial values that can be sold as individual products.

In some embodiments, the rejuvenated medium comprises glucose and fatty acids having a molecular weight greater than 150 Da, and is further subjected to pH neutralizing. In some embodiments, the rejuvenated medium is subjected to an anion exchange column. In some embodiments, the anion exchange column comprises at least one anion resin. By way of non-limiting example, the anion exchange column may comprise FPA55.

In some embodiments, a base may be added to neutralize the acidity of the rejuvenated medium. By way of non-limiting example, the base may be NaOH, sodium bicarbonate, potassium hydroxide, magnesium hydroxide, or calcium hydroxide. In some embodiments, the pH of the rejuvenated medium is adjusted to pH>6. By way of non-limiting example, the rejuvenated medium has a pH of about 7.

In some embodiments, the osmolarity of the rejuvenated medium is adjusted to be less than 360 milliosmoles per kilogram (mOsm/kg) of water. By way of non-limiting example, the rejuvenated medium has an osmolarity of about 280 mOsm/kg.

For any of the methods described above and herein, the recycled cell culture medium may be used to produce cultured meat.

Some aspects of the present disclosure provide a method for expanding cells in a bioreactor. This method comprises culturing tissues in a cell culture medium comprising nutrients and waste molecules; and recycling the cell culture medium according to the methods disclosed above and herein, to reduce the amount of waste molecules or remove the waste molecules from the medium. In some embodiments, the expanded cells are used to produce cultured meat.

Still some aspects of the present disclosure provide a method for reducing or removing waste products from a patient's blood. This method comprises obtaining blood from the patient using dialysis; filtering blood to obtain protein-free plasma containing waste products; and recycling the protein-free plasma according to the methods disclosed above and herein to reduce the amount of waste products or remove the waste products from the plasma.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A is a schematic diagram of rejuvenation system for recycling a cell culture medium.

FIG. 1B is a schematic diagram of an alternate rejuvenation system for recycling a cell culture medium using a centrifugation means 7.

FIG. 1C is a schematic diagram of rejuvenation system for recycling a cell culture medium further comprising an ultrafiltration unit 8 with a microfiltration means at 2.

FIG. 2A is a schematic diagram of cell culture system wherein the media is fresh.

FIG. 2B is a schematic diagram of rejuvenation system for recycling a cell culture medium wherein the media is a mixture of recycled and fresh media.

FIG. 2C is a schematic diagram of rejuvenation system wherein the media is a fully rejuvenated media.

FIG. 3 is a schematic diagram of an exemplary pilot scale production run of a immortalized fibroblasts, SCF-2 cell population. (1) Bioreactor, (2) ATF 50 kDa, (3) rejuvenation tank, (4) nanofiltration, (6) anion exchange column. The bioreactor had 270 L working volume (Solaris). The acid that was used is hydrochloric acid. The nanofiltration used DK membrane. The base that was used was sodium hydroxide.

FIG. 4A is a graph providing the rate of increase in immortalized fibroblasts, SCF-2 cell growth in a 2 L bioreactor. Squares represents a run used a fresh media in the perfusion. The maximal perfusion rate in this run was 26 L/day. Triangles represents a run used a mixture of 29% recycled media and 71% fresh media in the perfusion. The maximal perfusion rate of this run was 10 L/day. Circles represents a run used a mixture of 27% rejuvenated media and 73% fresh media.

FIG. 4B is a bar graph showing the reduction in glutamine, glutamate, glucose, lactate, ammonium, sodium and osmolarity during the rejuvenation phase.

FIG. 5A is a graph showing the perfusion rate (VVD) over time wherein the perfusion used 50% rejuvenated media from day 7. About 30 L, 80 L and 60 L of cells culture were harvested on days 6, 7 and 8, respectively.

FIG. 5B is a graph showing the perfusion rate (VVD) over time wherein the perfusion used 50% rejuvenated media from day 6. About 25 L and 36 L were harvested on days 6 and 7, respectively.

FIG. 5C is a graph showing the perfusion rate (VVD) over time wherein the perfusion used 50% rejuvenated media from day 8. About 50 L and 70 L were harvested on days 7 and 8, respectively.

FIG. 6 is a bar graph showing values for % amino acid retention for production pilot scale Run I. The retention of the process is defined as the percent of the amino acid concentration after the rejuvenation treatment over the concentration before the rejuvenation treatment. The amino acid concentrations were measured with an UPLC (Agilent).

FIG. 7 illustrates reduction measurement of the osmolarity (black bars), lactate (white bars) and ammonium (gray bars) using various types of resins. The screening test was performed in DMEM—high glucose medium, spiked with sodium lactate, ammonium chloride and sodium chloride. The initial lactate and ammonium concentrations were 45±15 mmole/L and 10 mmole/L ammonium, respectively. The osmolarity was adjusted to 430±30 mOsm/kg. The screening test was carried in multi-well plates (120 rpm) having a resin bed concentration of 10% w/v. Beds comprised of mixtures of resins were consisted of 55% anion type and 45% cation type.

FIG. 8 illustrates pH response at equilibrium to AmberLite FPC88 presence in DMEM —high glucose medium, spiked with 48 mmole/L lactate.

FIG. 9 is a bar graph illustrating nanofiltration removal to permeate of glutamine (diagonal stripes), glutamate (horizontal stripes), glucose (dotted), lactate (black), ammonium (white) and osmolarity (gray). The screening was carried over various nanofiltration membranes (lower horizontal axis) in three pH values: 2, 4 and 7 (upper horizontal axis). The feed was growth medium comprised of DMEM and was characterized by 30±13 mM lactate, 1.3±0.8 ammonium and 381±53 mOsm/kg. The operating pressure was 10.5 bars, and the recovery ratio was 70%.

FIG. 10 is a bar graph showing the nanofiltration removal to permeate over DL membrane at pH 7.5 (diagonal stipes), 6.1 (horizontal stripes), 3.9 (gray), 3.1 (black) and 2.0 (white). The feed was growth medium comprised of DMEM and was characterized by 26±1 mM lactate, 1.9±0.1 mM ammonium and 343±20 mOsm/kg. The operating pressure was 10.5 bars, and the recovery ratio was 70%.

FIG. 11 is a bar graph showing removal over nanofiltration and ion exchange treatments. The pH reduction to the required set-point was carried by with either pre-treatment with cation exchange column packed with AmberLite FPC88 resin, which pre-loaded with protons (gray bars), or by tittering with HCl. Nanofiltration process is given as black bars. A diafiltration was also executed, by pre-diluting the medium with deionized water prior the nanofiltration stage by 2-fold (white bars). Stacked bars are given as the removal of each element in the ion exchange pretreatment and the nanofiltration or the diafiltration stage. The feed was growth medium comprised of DMEM and characterized by 37±4 mM lactate, 3.5±1.7 mM ammonium and 383±61 mOsm/kg. The operating pressure was 10.5 bars, and the recovery ratio was 70%.

FIG. 12 is a bar graph depicting the bovine serum albumin content of waste media (black), ultrafiltration permeate (white, below the limit of detection), ultrafiltration followed by nanofiltration (gray) and nanofiltration without prior ultrafiltration step (diagonal black lines).

FIG. 13 is a graph depicting the flowrate through a microfilter (0.22 mm, PVDF, 8.5 cm²) of: waste media (black circles), media after ultrafiltration followed by nanofiltration (white squares) and media after nanofiltration without prior step of ultrafiltration (white triangles). The ultrafiltration was performed with a UF10 (TriSep™) membrane. The nanofiltration was carried out after reducing the pH to 2.8 by hydrochloric acid addition, with a DK membrane. The concentrate stream of each treatment (with and without prior step of ultrafiltration) was neutralized to pH 7.1, and diluted to 300 mOsm/kg.

### DETAILED DESCRIPTION

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terminology used herein is for the purpose of describing particular embodiments only and is not intended to be limiting of the invention.

As used herein, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, “devoid of,” “free” (as in “protein free”), “essentially devoid of,” or “essentially free”, means non-detectable or a small or insignificant amount of a contaminant. The term “non-detectable” is understood as based on standard methodologies of detection known in the art at the time of this application. In some embodiments, “a small amount” refers to less than 1% by weight.

As used herein the term “diminished” is understood to mean reduced amounts of a component (for example “waste material” or “protein”) in the medium, relative to the unprocessed medium. The term diminished is understood as based on standard methodologies of detection known in the art at the time of this application for the particular waste component. In some aspects the component may be reduced by about 80% to about 85%, or about 85% to about 90%, or about 90% to about 95%, or about 95% to about 99%, or about 99% to about 100% relative to the unprocessed medium. In some aspects the term diminished may also encompass “non-detectable” or a small or insignificant amount of a component. In such particular aspects where the component is “non-detectable” the term may be used interchangeably to mean “devoid of”, “free” (as in “protein free”), “essentially devoid of,” or “essentially free”.

As used herein, the terms “waste material(s)” and “waste molecule(s)” are interchangeable. These are any materials/molecules that interfere with desired growth and/or desired differentiation of the cells that are cultured in a cell culture medium, e.g., inhibit cell growth and/or differentiation or induce cell death. These materials/molecules are usually selected amongst minerals (mainly sodium salts) and small molecules (low molecular weight molecules). By way of non-limiting examples, the waste materials/molecules include, but are not limited to, ammonia, lactate, toxins, sodium salts, alanine, glutamic acid, aspartic acid, ammonium, reactive oxygen and nitrogen species.

As used herein, the term “medium” or “cell culture medium” encompasses any such medium as known in the art, including cell suspensions, blood and compositions comprising ingredients of biological origin. Such media and cultures may contain cells (mammalian cells, chicken cells, crustacean cells, fish cells and other cells), blood components, nutrients, supplements and feeds, amino acids, peptides, proteins and growth factors (such as albumin, catalase, transferrin, fibroblast growth factor (FGF), and others), vitamins, polyamines, sugars, carbohydrates, lipids, nucleic acids, hormones, fatty acids, trace materials, certain salts (such as potassium salts, calcium salts, magnesium salts), as well as waste materials such as ammonia, lactate, toxins and sodium salts. The medium is typically an aqueous based solution that promotes the desired cellular activity, such as viability, growth, proliferation, differentiation of the cells cultured in the medium. The pH of a culture medium should be suitable to the microorganisms that will be grown. Most bacteria grow in pH 6.5-7.0 while most animal cells thrive in pH 7.2-7.4.

As used herein, “hollow fibers” are elongated tubular membranes which may be specifically prepared from polymeric materials or other materials, or alternatively, obtained commercially. By way of non-limiting examples, hollow fibers and systems employing the same that can be used, modified or adapted for use in accordance with the present disclosure include those disclosed in U.S. Pat. Nos. 9,738,918; 9,593,359; 9,574,977; 9,534,989; 9,446,354; 9,295,824; 8,956,880; 8,758,623; 8,726,744; 8,677,839; 8,677,840; 8,584,536; 8,584,535; and 8,110,112, each of which is incorporated herein by reference.

“Diafiltration (DF)” means the process of diluting a concentrate and reapplying the diluted concentrate to a membrane.

“Microfiltration (MF)” means the process of delivering a liquid/suspension to a membrane with a pore size of 0.1 to 10 μm.

“Nanofiltration (NF)” means the process of delivering a liquid/suspension to membranes with a pore size of 10 to 100 A, including the use of membranes that are charged such as negatively charged membranes.

“Ultrafiltration (UF)” means the process of delivering a liquid/suspension to a membrane with a pore size of 30 to 1,000 A.

As used herein the term “method” or “methods” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The present disclosure provides, in part, improved systems or methods of effectively filtering waste materials from cell culture media and recycling the media for large scale biological manufacturing of cells, proteins, or vaccines. The systems and methods disclosed above and herein separate essential materials from waste materials in liquid media, and rejuvenating and recycling the media for continuous use, thereby provide cost effective cell culture media. While the systems or methods may be used for treating a vast array of liquid formulations or compositions, the present disclosure focuses on using these systems and methods as efficient and simple ways to separate waste components from essentials of cell culture media and recycle the media for continuous use.

Accordingly, one aspect of the present disclosure provides a system for recycling a cell culture medium. In some aspects the system comprises a bioreactor and a rejuvenation system. In some aspects the bioreactor and the rejuvenation system are in communication. In some aspect the bioreactor and the rejuvenation system may operate as a loop for example, the waste medium from the bioreactor flows into the rejuvenation system and the rejuvenated medium is fed back into the bioreactor. In some aspects the rejuvenation system is independent of the bioreactor and the bioreactor comprises a mean for harvesting the waste medium for further processing in a rejuvenation system. In some aspects the disclosed rejuvenation system comprises one or more if a filtration (MF or UF) and/or centrifugation means, an ultrafiltration (UF) means, an acidification means, an osmolarity adjustments means, a nanofiltration (NF) means, a neutralization means. In some aspects the system may further comprise means for harvesting one or more of cells, products and media components for further processing.

In some aspects the system comprises means for removing a cell culture medium from a bioreactor, means for filtering the cell culture medium, thereby obtaining a waste medium and a concentrate medium. The waste medium comprises at least one waste material and is essentially devoid of any cells and large proteins. While the concentrate medium is circulated back into the bioreactor, the waste medium is further processed.

To further process the waste medium, the system comprises means for acidifying the waste medium, and means for subjecting the acidified waste medium to nanofiltration, thereby removing the at least one waste material from the waste medium and obtaining a rejuvenated medium that is diminished for at least one waste material. The rejuvenated medium may be essentially devoid of at least one waste material. In some aspects the system may optionally comprise a means for subjecting the waste medium to ultrafiltration prior acidification and nanofiltration. The rejuvenated medium is further processed and circulated back into the bioreactor, thereby the cell culture medium is recycled.

In some embodiments, the cell culture medium comprises one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials. By way of non-limiting example, the cell culture medium may comprise blood cells.

In some embodiments, the waste materials are any materials that interfere with desired growth and/or desired differentiation of cells cultured in the cell culture medium. For instance, the waste materials may inhibit cell growth and/or differentiation or induce cell death. In some embodiments, the waste material(s) include, but are not limited to, ammonia, lactate, toxins, sodium salts, alanine, glutamic acid, aspartic acid, ammonium, reactive oxygen and nitrogen species. By way of non-limiting example, the at least one waste material may comprise ammonia, ammonium, and/or lactate.

In some embodiments, the waste materials have a molecular weight of no greater than 60 kDa, e.g., no greater than 55 kDa, no greater than 50 kDa, no greater than 45 kDa, no greater than 40 kDa, no greater than 35 kDa, no greater than 30 kDa, no greater than 25 kDa, no greater than 20 kDa, no greater than 15 kDa, or no greater than 10 kDa.

In some embodiments, a culture medium of cells or tissues is filtered and recycled, wherein tissues are cultured for antibody production. Trough the filtration and recycling, at least one waste materials are removed/diminished from the culture medium, while the produced (or secreted) antibodies are retained in the culture medium.

In some embodiments, a culture medium of cells or tissues is filtered and recycled, wherein tissues are cultured for cultured meat production in at least one container, e.g., a bioreactor. Through the filtration and recycling, at least one waste material that interfere with the proper growth of the cultured meat and/or that cause cell death is removed/diminished from the culture medium, while nutrients needed for the proper growth of the cultured meat are retained in the culture medium.

In some embodiments, the filtering means is a normal flow filtration (NFF) system. In some embodiments, the filtering means is a tangential flow filtration (TFF). In some aspects the filtering means is a TFF for example an alternate tangential flow (ATF) system. In some aspects the ATF comprises a microscale filtration system, for example when harvesting cells. In some aspects the ATF comprises a ultra-grade filtration system. In some aspects the ATF comprises at least one hollow fiber. In some embodiments, the porosity profile of the hollow fiber walls is configured to retain cells with microfiltration scale retention capacity. For example, the porosity profile is configured to retain cells and suspended solids. In some embodiments, the porosity profile of the hollow fiber walls is configured to have ultrafiltration scale retention capacity. In some aspects the porosity profile is configured to retain cells, viruses, certain biomolecules like proteins with ultrafiltration scale retention capacity. In some embodiments, the porosity profile of the hollow fiber walls is configured to provide an average pore size and pore density that only permits passage of molecules that are smaller than 60 kDa.

In some embodiments, to permit flow of the culture medium along the hollow fiber, each hollow fiber may be configured to have an internal diameter of at least 0.1 mm, or at least 0.5 mm, or at least 0.75 mm, up to 5 mm. In some embodiments, each hollow fiber is configured to have an internal diameter that permit flow of cells and other culture components having diameters of between 5 and 20 micrometers.

The porous hollow fiber walls act to prevent nutrients and other essential materials from crossing through. This is achieved by a porosity profile selected to provide optimal pore size and pore density. Each hollow fiber may be selected to have the same porosity profile. While the pores diameters (cut-off size) may not be constant, the pores diameter should on average be selected to prevent passage of high molecular weight materials, while permitting facile and efficient passage of small molecules, i.e., low molecular weight waste materials. In some embodiments, the cut-off pore size is no greater than or smaller than 60 kDa (and different from or greater than 0 kDa). In some embodiments, the average pore diameter is such that a material having a molecular weight of between 10 and 60 kDa can pass through. In some embodiments, the average pore diameter is such that a material having a molecular weight of between 10 and 20 kDa, between 10 and 25 kDa, between 10 and 30 kDa, between 10 and 35 kDa, between 10 and 40 kDa, between 10 and 45 kDa, between 10 and 50 kDa, between 10 and 55 kDa, between 15 and 60 kDa, between 20 and 60 kDa, between 25 and 60 kDa, between 30 and 60 kDa, between 35 and 60 kDa, between 40 and 60 kDa, between 45 and 60 kDa or between 50 and 60 kDa can pass through. In some embodiments, the cutoff pore diameter is no greater than 10 kDa.

The pore density, namely the number of pores per unit surface area of the inner fiber wall, may be varied according to the porosity of the hollow fibers. In some embodiments, at least 10% of the inner fiber walls are porous. That is, the pore density is at least 10% of the wall surface of each hollow fiber.

In some embodiments, the filtering means comprises density centrifugation. By way of non-limiting example, the centrifuge is a continuous disc stack hermetic centrifuge operating at 8400×g. In some embodiments, the centrifuge operates at a speed between 1000 to 2000×g, between 1000 to 6000×g, between 1000 to 8000×g, between 1000 to 10,000×g, or between 1000 to 20,000×g.

The cell culture medium comprises nutrients, essential materials, and waste materials, wherein separation is desired to remove the waste materials from the medium. The essential materials and nutrients are differentiated from the waste materials according to their sizes in that the waste materials are materials having molecular weights below (or no greater than) 60 kDa, whereas the essential materials and nutrients are materials having molecular weights greater than or equal to 61 kDa.

For the cell culture recycling systems disclosed above and herein, the concentrate medium comprises cells and essential materials for cell growth and/or differentiation and is circulated back into the bioreactor after going through the filtering means.

While the concentrate medium is circulated back into the bioreactor, the waste medium goes through further processing. Further processing may include one or more of: ultrafiltration, nanofiltration, adjustment of osmolarity, and/or adjustment of pH (e.g., acidification and/or neutralization).

In some embodiments, for example when the ATF comprises microscale filtration (for example with a 0.22 um cut-off), there may be a need to remove biomolecules like proteins from the waste medium prior to acidification. In such and other similar instances, it may be desirable to add an ultrafiltration means prior to acidification.

In some embodiments, the means for acidifying the waste medium comprises subjecting the waste medium to a cation exchange column. In some embodiments, the cation exchange column comprises at least one cation resin. By way of non-limiting example, the cation exchange column may comprise AmberLite FPC88.

In some embodiments, the means for acidifying the waste medium comprises adding an acid thereto. In some embodiments, an acid may be added to acidify the waste medium. By way of non-limiting example, the acid may be HCl, sulfuric acid, nitric acid, phosphoric acid, carbonic acid, citric acid, or acetic acid. In some embodiments, the acidified waste medium has a pH value of less than 4. In some embodiments, the acidified waste medium has a pH of 4.5, or 4, or 3.5 or 3 or 2.5, or 2, or 1.5 or any intermediate pH. By way of non-limiting example, the acidified waste medium has a pH value of about 2.

The acidified waste medium then goes through nanofiltration to further separate the waste materials and remaining essential materials in the medium. In some embodiments, the nanofiltration is also performed as a diafiltration mode which involves pre-diluting the acidified waste medium with deionized water before the nanofiltration. In some embodiments, the nanofiltration has a molecular weight cutoff of from about 150 to about 300 Da, e.g., from about 150 to about 200 Da, from about 150 to 250 Da, from about 200 to about 250 Da, from about 200 to about 300 Da, from about 250 to about 300 Da.

In some embodiments, the waste materials are recovered from the acidified waste medium post nanofiltration. The waste materials include, but are not limited to, ammonia, ammonium, and lactate. In some embodiments, these components are further isolated and recovered individually. These recovered individual components such as ammonia, ammonium salts, and lactate may possess commercial values that can be sold as individual products.

After the nanofiltration, a rejuvenated medium is obtained, which comprises glucose and fatty acids having a molecular weight of greater than 150 Da. Such rejuvenated medium is further processed by means of neutralizing the pH thereof. In some embodiments, the neutralizing means comprises subjecting the rejuvenated medium to an anion exchange column. In some embodiments, the anion exchange column comprises at least one anion resin. By way of non-limiting example, the anion exchange column may comprise FPA55, IRA410, IRA67, or HPR4800. By way of non-limiting example, the anion exchange column may comprise FPA55.

In some embodiments, a base may be added to neutralize the acidity of the rejuvenated medium. By way of non-limiting example, the base may be NaOH, sodium bicarbonate, potassium hydroxide, magnesium hydroxide, or calcium hydroxide. In some embodiments, the pH of the rejuvenated medium is adjusted to pH>6. By way of non-limiting example, the rejuvenated medium has a pH of about 7.

In some embodiments, the osmolarity of the rejuvenated medium is adjusted to be less than 360 milliosmoles per kilogram (mOsm/kg) of water. By way of non-limiting example, the rejuvenated medium has an osmolarity of about 280 mOsm/kg.

In some embodiments, after pH neutralization and subjection to the anion exchange column, the rejuvenated medium is diluted with water before being circulated back into the bioreactor. After the filtration and recycling, the system disclosed above and herein provides a recycled medium that comprises less than 30%, e.g., less than 20%, less than 10%, less than 5%, less than 2% or any intermediate, smaller or larger percentage value of waste molecules compared to the amount of waste molecules in the culture medium entering the system. In some embodiments, the recycled medium comprises more than 60%, e.g., more than 70%, more than 80%, more than 90%, more than 95% or any intermediate, smaller or larger percentage value of selected nutrients or other essential materials compared to the amount of the selected nutrients or other essential materials in the culture medium entering the system.

In some embodiments, the system may also comprise a means for adding fresh culture medium to the bioreactor in addition to the concentrate medium and rejuvenated medium. In some aspects the fresh culture medium may amount to about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 100% of the total added media.

In some embodiments, the cell culture medium is a suspension containing animal cells that is perfused using a pump into the filtering means (e.g., hollow fiber). The pump may be a positive displacement pump that works to push the suspension through the filtering means or to alternate between pushing the suspension into the filtering means and drawing it out into the bioreactor. In some embodiments, the cells are retained with the nutrients due to their sizes. In some embodiments, animal cells are retained in the bioreactor using a filter and only the culture medium is introduced to the filtering means.

For any of the systems of recycling cell culture media described above and herein, biomass is expanded in the cell culture medium to produce edible/cultured meat. At high cell densities, the cell growth can be limited by the lack of nutrients or by the presence of produced metabolites that have inhibitory effect. Therefore, at high cell densities, continuous supplement of nutrients and reduction of inhibitors is a critical strategy to maintain the log phase of the cells. Feeding fresh media in a perfusion process can supply nutrients and dilute the inhibitors concentration at the bioreactor, but this requires large amounts of fresh media and is too expensive for a food technology process. Recycling the media at a perfusion process can supply nutrients that were not fully consumed by the cells, and to reduce the required volume of fresh media to some extent. However, inhibitors will be recycled to the bioreactor as well. Media rejuvenation and rejuvenation systems integrated with bioreactors can be optimized to selectively remove these inhibitory metabolites, while retaining essential nutrients in the media. This system provides cost effective cell culture media for example for mass production of edible/cultured meat.

Another aspect of the present disclosure provides a method for recycling a cell culture medium. Such method comprises removing a cell culture medium from a bioreactor; filtering the cell culture medium, thereby obtaining a waste medium and a concentrate medium; optionally subjecting the waste medium to ultrafiltration; acidifying the waste medium; and subjecting the acidified waste medium to nanofiltration, thereby removing the at least one waste material from the waste medium and obtaining a rejuvenated medium. In this method, upon filtration, the waste medium comprises at least one waste material and is essentially devoid of any cells and large proteins, and the concentrate medium is diminished for or is essentially devoid of at least one waste material. While the concentrate medium is circulated back into the bioreactor, the waste medium is further processed.

In some embodiments, the cell culture medium comprises one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials. By way of non-limiting example, the cell culture medium may comprise blood cells.

In some embodiments, the waste materials are any materials that interfere with desired growth and/or desired differentiation of cells cultured in the cell culture medium. For instance, the waste materials may inhibit cell growth and/or differentiation or induce cell death. In some embodiments, the waste material(s) include, but are not limited to, ammonia, lactate, toxins, sodium salts, alanine, glutamic acid, aspartic acid, ammonium, reactive oxygen and nitrogen species. By way of non-limiting example, the at least one waste material may comprise ammonia, ammonium, and/or lactate.

In some embodiments, the waste materials have a molecular weight of no greater than 60 kDa, e.g., no greater than 55 kDa, no greater than 50 kDa, no greater than 45 kDa, no greater than 40 kDa, no greater than 35 kDa, no greater than 30 kDa, no greater than 25 kDa, no greater than 20 kDa, no greater than 15 kDa, or no greater than 10 kDa.

For the methods of recycling a cell culture medium disclosed above and herein, the culture medium of cells or tissues is filtered and recycled, wherein tissues are cultured for antibody production. Through the filtration and recycling, the waste materials are removed/diminished from the culture medium, while the produced (or secreted) antibodies are retained in the culture medium.

For the methods of recycling a cell culture medium disclosed above and herein, the culture medium of cells or tissues is filtered and recycled, wherein tissues are cultured for cultured meat production in at least one container, e.g., a bioreactor. Through the filtration and recycling, the waste materials that interfere with the proper growth of the cultured meat and/or that cause cell death are removed/diminished from the culture medium, while nutrients needed for the proper growth of the cultured meat are retained in the culture medium.

To filter the cell culture medium that exit the bioreactor, at least one hollow fiber may be used. In some embodiments, the porosity profile of the hollow fiber walls is configured to provide an average pore size and pore density that only permits passage of molecules that are smaller than 60 kDa.

To permit flow of the culture medium along the hollow fiber, each hollow fiber is configured to have an internal diameter of at least 0.1 mm, or at least 0.5 mm, or at least 0.75 mm, up to 5 mm. In some embodiments, each hollow fiber is configured to have an internal diameter that permit flow of cells and other culture components having diameters of between 5 and 20 micrometers.

The porous hollow fiber walls act to prevent nutrients and other essential materials from crossing through. This is achieved by a porosity profile selected to provide optimal pore size and pore density. Each hollow fiber may be selected to have the same porosity profile. While the pores diameters (cut-off size) may not be constant, the pores diameter should on average be selected to prevent passage of high molecular weight materials, while permitting facile and efficient passage of small molecules, i.e., low molecular weight waste materials. In some embodiments, the cut-off pore size is no greater than or smaller than 60 kDa (and different from or greater than 0 kDa). In some embodiments, the average pore diameter is such that a material having a molecular weight of between 10 and 60 kDa can pass through. In some embodiments, the average pore diameter is such that a material having a molecular weight of between 10 and 20 kDa, between 10 and 25 kDa, between 10 and 30 kDa, between 10 and 35 kDa, between 10 and 40 kDa, between 10 and 45 kDa, between 10 and 50 kDa, between 10 and 55 kDa, between 15 and 60 kDa, between 20 and 60 kDa, between 25 and 60 kDa, between 30 and 60 kDa, between 35 and 60 kDa, between 40 and 60 kDa, between 45 and 60 kDa or between 50 and 60 kDa can pass through. In some embodiments, the cutoff pore diameter is no greater than 10 kDa.

The pore density, namely the number of pores per unit surface area of the inner fiber wall, may be varied according to the porosity of the hollow fibers. In some embodiments, at least 10% of the inner fiber walls are porous. That is, the pore density is at least 10% of the wall surface of each hollow fiber.

The cell culture medium comprises nutrients, essential materials, and waste materials, wherein separation is desired to remove/reduce the waste materials from the medium. The essential materials and nutrients are differentiated from the waste materials according to their sizes in that the waste materials are materials having molecular weights below (or no greater than) 60 kDa, whereas the essential materials and nutrients are materials having molecular weights greater than or equal to 61 kDa.

For the cell culture recycling methods disclosed above and herein, the concentrate medium comprises cells and essential materials for cell growth and/or differentiation and is circulated back into the bioreactor for continuous use. While the concentrate medium is circulated back into the bioreactor, the waste medium goes through further processing. Further processing may include one or more of: ultrafiltration, nanofiltration, adjustment of osmolarity, and/or adjustment of pH (e.g., acidification and/or neutralization).

In some embodiments, for example when the ATF comprises microscale filtration (for example with a 0.22 μm cut-off), there may be a need to remove biomolecules like proteins from the waste medium prior to acidification. In such and other similar instances, it may be desirable to add an ultrafiltration means prior to acidification.

In some embodiments, the waste medium is subjected to a cation exchange column. In some embodiments, the cation exchange column comprises at least one cation resin. By way of non-limiting example, the cation exchange column may comprise AmberLite FPC88.

In some embodiments, the waste medium is subjected to addition of an acid for acidification. By way of non-limiting example, the acid may be HCl, sulfuric acid, nitric acid, phosphoric acid, carbonic acid, citric acid, or acetic acid. In some embodiments, the acidified waste medium has a pH value of less than 4. By way of non-limiting example, the acidified waste medium has a pH value of about 2.

The acidified waste medium then goes through nanofiltration to further separate the waste materials and remaining essential materials in the medium. In some embodiments, the nanofiltration is also performed as a diafiltration mode which involves pre-diluting the acidified waste medium with deionized water before the nanofiltration. In some embodiments, the nanofiltration has a molecular weight cutoff of from about 150 to about 300 Da, e.g., from about 150 to about 200 Da, from about 150 to 250 Da, from about 200 to about 250 Da, from about 200 to about 300 Da, from about 250 to about 300 Da.

In some embodiments, the methods described above and herein may further comprise recovering the waste materials from the acidified waste medium post nanofiltration; isolating the components of the waste materials; and recovering the individual components. In some embodiments, the waste materials comprise ammonia, ammonium, and/or lactate, and the recovered individual components may possess commercial values that can be sold as individual products.

After the nanofiltration, a rejuvenated medium is obtained, which comprises glucose and fatty acids having a molecular weight of greater than 150 Da. Such rejuvenated medium is further subjected to pH neutralizing. In some embodiments, the rejuvenated medium is subjected to an anion exchange column. In some embodiments, the anion exchange column comprises at least one anion resin. By way of non-limiting example, the anion exchange column may comprise FPA55, IRA410, IRA67, or HPR4800. By way of non-limiting example, the anion exchange column may comprise FPA55.

In some embodiments, a base may be added to neutralize the acidity of the rejuvenated medium. By way of non-limiting example, the base may be NaOH, sodium bicarbonate, potassium hydroxide, magnesium hydroxide, or calcium hydroxide. In some embodiments, the pH of the rejuvenated medium is adjusted to pH>6. By way of non-limiting example, the rejuvenated medium has a pH of about 7.

In some embodiments, the osmolarity of the rejuvenated medium is adjusted to be less than 360 milliosmoles per kilogram (mOsm/kg) of water. By way of non-limiting example, the rejuvenated medium has an osmolarity of about 280 mOsm/kg.

In some embodiments, after pH neutralization and subjection to the anion exchange column, the rejuvenated medium is diluted with water before being circulated back into the bioreactor. After the filtration and recycling, the method disclosed above and herein provides a recycled cell culture medium that comprises less than 300, e.g., less than 20%, less than 10%, less than 5%, less than 2% or any intermediate, smaller or larger percentage value of waste molecules compared to the amount of waste molecules in the cell culture medium prior to filtration and recycling. In some embodiments, the recycled cell culture medium comprises more than 60%, e.g., more than 70%, more than 80%, more than 90%, more than 95% or any intermediate, smaller or larger percentage value of selected nutrients or other essential materials compared to the amount of the selected nutrients or other essential materials in the cell culture medium prior to filtration and recycling.

In some embodiments, the method may include adding fresh culture medium to the bioreactor in addition to the concentrate medium and rejuvenated medium. In some aspects the fresh culture medium may amount to about 10% to about 20%, or about 20% to about 30%, or about 30% to about 40%, or about 40% to about 50%, or about 50% to about 60%, or about 60% to about 70%, or about 70% to about 80% of the total added media. In some aspects the rejuvenated medium may comprise fresh and recycled media.

In some embodiments, the cell culture medium is a suspension containing animal cells that is perfused using a pump into the filtering means (e.g., hollow fiber). The pump may be a positive displacement pump that works to push the suspension through the filtering means or to alternate between pushing the suspension into the filtering means and drawing it out into the bioreactor. In some embodiments, the cells are retained with the nutrients due to their sizes. In some embodiments, animal cells are retained in the bioreactor using a filter and only the culture medium is introduced to the filtering means.

For any of the methods of recycling cell culture media described above and herein, the recycled cell culture medium may be used to produce cultured meat. In some embodiments, biomass is expanded in the cell culture medium to produce edible/cultured meat. These methods provide cost effective cell culture media for mass production of edible/cultured meat. Lack of nutrients and presence of produced metabolites that have inhibitory effect in used media can be a limiting factor for attaining high cell densities required for producing cultured meat. Continuous supplement of nutrients and reduction of inhibitors is a critical strategy to maintain high culture densities. Feeding fresh media in a perfusion process can supply nutrients and dilute the inhibitors concentration at the bioreactor, but this requires large amounts of fresh media and is too expensive for a food technology process. Recycling the media at a perfusion process can supply nutrients that were not fully consumed by the cells, and to reduce the required volume of fresh media to some extent. However, inhibitors will be recycled to the bioreactor as well. Media rejuvenation and rejuvenation systems integrated with bioreactors can be optimized to selectively remove these inhibitory metabolites, while retaining essential nutrients in the media.

Some aspects of the present disclosure provide a method for expanding cells in a bioreactor. This method comprises culturing tissues in a cell culture medium comprising nutrients and waste molecules; and recycling the cell culture medium according to the methods disclosed above and herein, to reduce the amount of waste molecules or remove the waste molecules from the medium. In some embodiments, the expanded cells are used to produce cultured meat.

Still some aspects of the present disclosure provide a method for reducing or removing waste products from a patient's blood. This method comprises obtaining blood from the patient using dialysis; filtering blood to obtain protein-free plasma containing waste products; and recycling the protein-free plasma according to the methods disclosed above and herein to reduce the amount of waste products or remove the waste products from the plasma.

The following examples are offered by way of illustration and not by way of limitation.

### Example 1: Cell Culture Recycling/Rejuvenation System

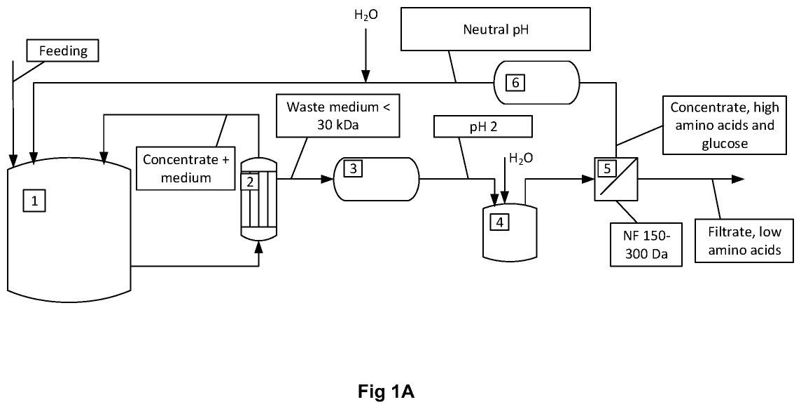

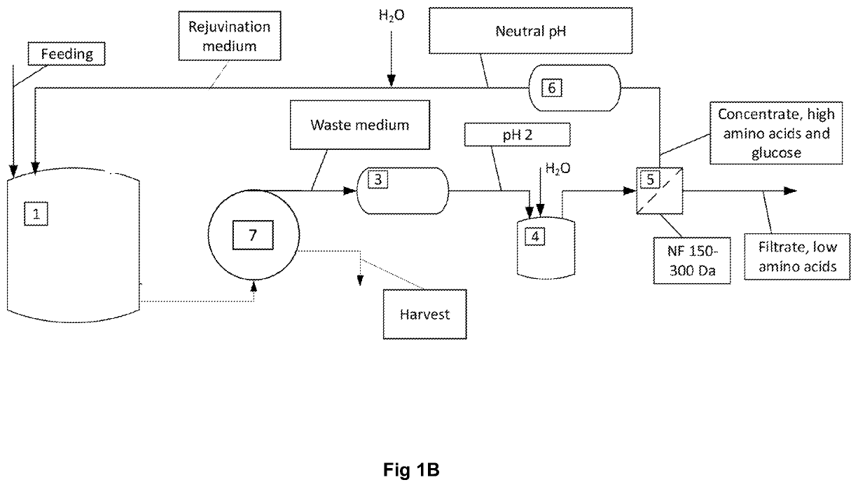

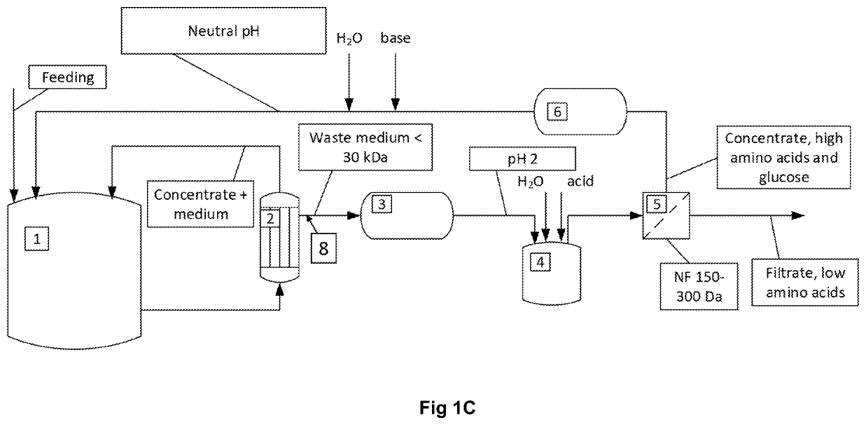

A system or process of filtering and recycling a cell culture medium is illustrated in FIG. 1A or FIG. 1B. Such rejuvenation system can be used for filtering and recycling different types of cell culture media. For example, a suspension culture of cells/tissues useful for cellular therapy, protein or vaccine production, tissue transplantation, or cultured meat production may go through this system or process for filtration and recycling.

FIG. 1A is a schematic diagram of the rejuvenation system. As an overview, a waste medium from a bioreactor is filtrated through a hollow fiber holding a 30 kDa MWCO. The hollow fiber permeates flows to the rejuvenation system. The waste medium is first acidified by flowing in a cation exchange column and/or by adding an acid. After the waste medium is acidified, it enters to the nanofiltration stage (150-300 MWCO), and a nanofiltration retentate stream is recirculated back to the bioreactor after neutralizing and diluting. A diafiltration mode can be used by introducing water prior the nanofiltration stage.

More specifically, as illustrated in FIG. 1A, the rejuvenation system comprises bioreactor 1 for culturing the cells or tissues therein, a delivery means configured to deliver or feed a perfusion solution or cell culture medium to the bioreactor. The feeding is optionally and preferably continuous.

The rejuvenation system also comprises means for removing a cell culture medium from bioreactor 1, followed by means for filtering the cell culture medium (e.g., hollow fiber or centrifuge 2 or 7 in FIGS. 1A and 1B respectively), thereby obtaining a waste medium and a concentrate medium. The waste medium contains waste material(s) that interfere with desired cell growth and/or differentiation and is essentially devoid of cells or large proteins, whereas the concentrate medium contains cells and other essential material(s) for cell growth and/or differentiation.

Hollow fiber 2 comprises porous walls that act to prevent nutrients and other essential materials from crossing through. This is achieved by a porosity profile selected to provide optimal pore size and pore density. Each hollow fiber may be selected to have the same porosity profile. While the pores diameters (cut-off size) may not be constant, the pores diameter should on average be selected to prevent passage of high molecular weight materials, while permitting facile and efficient passage of small molecules, i.e., low molecular weight waste materials. In this system, the porosity profile of the hollow fiber walls is configured to provide an average pore size and pore density that only permits passage of molecules that are smaller than 30 kDa. As such, the waste medium contains waste material(s) smaller than 30 kDa.

After the filtration, the concentrate medium is circulated back into the bioreactor, while the waste medium is subjected to further processes. As illustrated therein, the system further comprises means for acidifying the waste medium (e.g., cation exchange column 3) and means for subjecting the acidified waste medium to nanofiltration 5. Before the nanofiltration, the waste medium has a pH value of less than 4, preferably about 2. The nanofiltration is also performed as a diafiltration mode which involves pre-diluting the acidified waste medium with deionized water before the nanofiltration (see, rejuvenation tank 4).

In some instances, for instance when cells are harvested using a micro-scale filtration means or low speed centrifugation or when the ATF uses a micro-scale filtration, an ultrafiltration step 8 (FIG. 1C) may be optionally added before acidification of the waste medium to separate the proteins in the waste medium.

As illustrated, nanofiltration 5 has a molecular weight cutoff of from about 150 to about 300 Da. After the nanofiltration, the waste materials (i.e., filtrate) are separated from the remaining essential materials in the waste medium, and a rejuvenated medium is obtained that is diminished in waste materials.

The filtrate may contain ammonia, ammonium salts, lactate, and/or amino acids of low molecular weight. It may undergo further processes to isolate and recover individual components. The recovered individual components may possess commercial values that can be sold as individual products.

The rejuvenated medium may contain amino acids of high molecular weight and glucose and is further neutralized by flowing through anion exchange column 6 and subsequently circulated back into bioreactor 1 after dilution with water.

FIG. 1B is a schematic diagram of another rejuvenation system. As an overview, a waste medium from bioreactor 1 is filtrated through continuous disc stack centrifuge 7 at 8400×g or faster. The light phase of the centrifuge is composed of the waste medium that flows to the rejuvenation system, while the solid phase is continuously harvested. In some instances, before acidification the waste medium is subjected to ultrafiltration (see FIG. 1C but here the cells are harvested using a centrifuge) to separate the proteins in the waste medium. The waste medium is then acidified by flowing in cation exchange column 3 and/or by adding an acid. After the waste medium is acidified, it enters the nanofiltration stage 5 (150-300 MWCO), and a nanofiltration retentate stream is recirculated back to bioreactor 1 after neutralizing and diluting. A diafiltration mode can be used by introducing water prior the nanofiltration stage.

Centrifuge 7 comprises rapidly rotating drum that uses centrifugal forces to separate light from heavy materials. Centrifuge type and speed may be selected to support certain flow rates and certain rotation speeds that impart centrifugal forces on components within the media. In this system, rotation speed of 8400xg is configured to separate cells and large protein aggregates ranging from 30 to 150 kDa. As such, the waste medium contains waste material(s) smaller than 30 kDa.

After the filtration, the heavy phase can be harvested or circulated back to the bioreactor as concentrated medium, while the waste medium is subjected to further processes. As illustrated therein, the system further comprises means for acidifying the waste medium (e.g., cation exchange column 3) and means for subjecting the acidified waste medium to nanofiltration 5. Before the nanofiltration, the waste medium has a pH value of less than 4, preferably about 2. The nanofiltration is also performed as a diafiltration mode which involves pre-diluting the acidified waste medium with deionized water before the nanofiltration (see, rejuvenation tank 4).

### Example 2: The Rejuvenation Process Integrated in a Cell Growth Bioreactor System

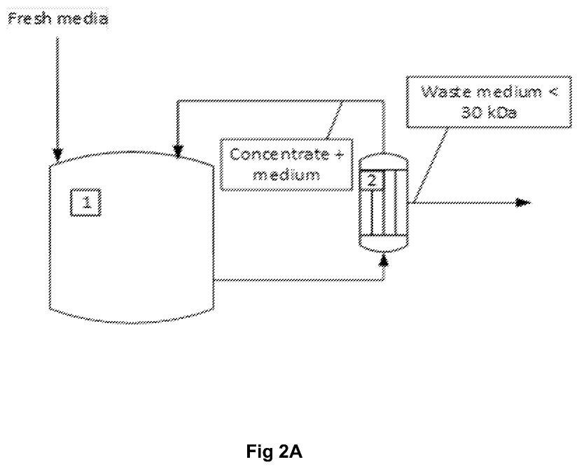

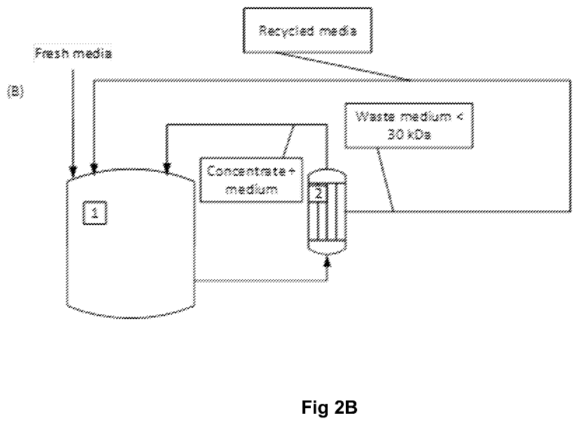

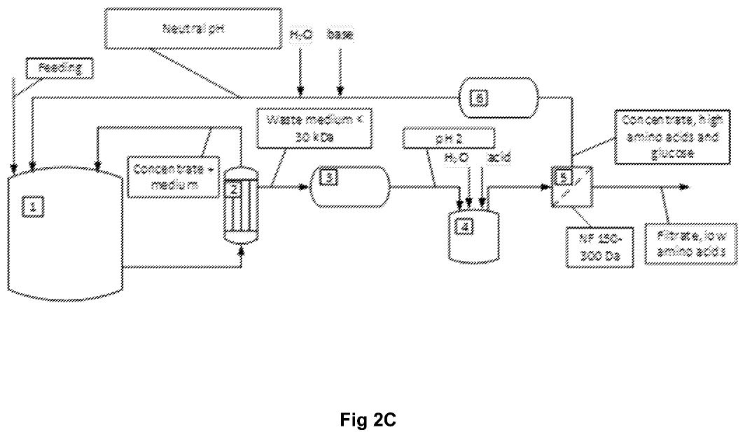

In order to estimate the rejuvenation effect on cell growth in a bioreactor and ensure the rejuvenation process is applicable, a cultured cell population was grown in a 2 L bioreactor (Twin B, Sartorius, cell population was immortalized fibroblasts, SCF-2) using DMEM media and was tested with an integrated rejuvenation system. The cell growth runs were initially started with a fed-batch phase. At the fed-batch phase, the bioreactor was fed by essential nutrients that were consumed by the cells (e.g., glucose and glutamine). The fed-batch phase was followed by a perfusion phase. At the perfusion phase, the bioreactor media, which contained produced metabolites (such as lactate and ammonium), was replaced by different media, while retaining the cells in the bioreactor. The perfusion enables growth at high cell densities due to reduction of the inhibitory metabolites and by ensuring sufficient source of nutrients. Three different media sources were used: fresh media (FIG. 2A), a mixture of recycled media and fresh media (FIG. 2B), and rejuvenated media (FIG. 2C).

The cells were retained to the process at the perfusion phase using an alternating tangential flow (ATF, Repligen) filtration system (30 kDa). The waste medium, which contained molecules smaller than the ATF cutoff, may be recycled or rejuvenated as presented in FIG. 2B and FIG. 2C, respectively. When rejuvenated (FIG. 2C), the perfusate was fed to a rejuvenation holding tank after pH adjustment to pH 2. FPC88 was initially used as cation exchange bed to adjust the pH to a value of pH 2. The cation exchange process was followed by nanofiltration process, used a DK membrane (Suez). The operating pressure at the nanofiltration was 10 bar, and the recovery was 71%. The acidified bioreactor waste was then transferred from the rejuvenation holding tank to the nanofiltration system. More specifically as shown in FIG. 2C, the waste medium from the bioreactor was filtrated through an hollow fiber holding a 30 kDa MWCO. The hollow fiber permeate was channeled to the rejuvenation system. The waste was first acidified by flowing in a cation exchange column and/or by adding acid. After the waste was acidified, it entered the NF stage (150-300 MWCO). A diafiltration mode was used by introducing water prior the NF stage. The concentrate, which was rich in amino acids and glucose, was then re-balanced in terms of neutral pH (pH 7) and osmolarity (300 mOsm/kg) using sodium hydroxide and deionized water, respectively and the NF retentate stream was recirculated back to the bioreactor system.

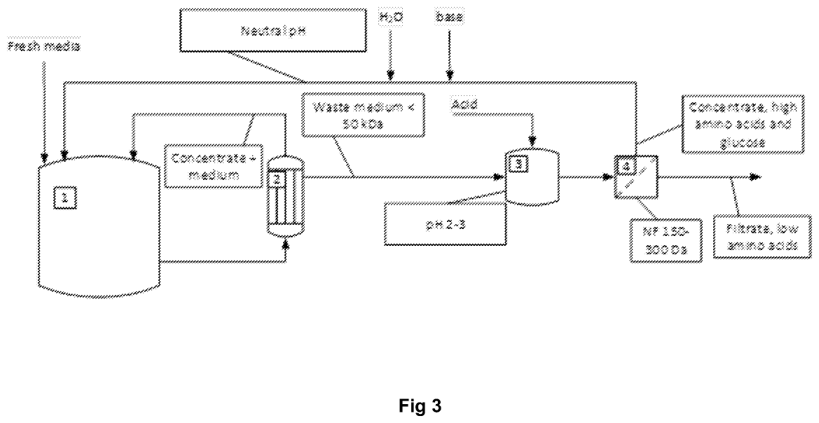

FIG. 3 is a schematic figure of a 4^{th} exemplary pilot scale system that may optionally be used in some cases to produce immortalized fibroblasts, SCF-2 cells integrated with a rejuvenation system as in FIG. 2C. Here a different cut-off filtration membrane was used, and it omits the cation exchange step. This platform used only hydrochloric acid to adjust the pH prior the nanofiltration stage. However, in the present example, the configuration in FIG. 2C was used.

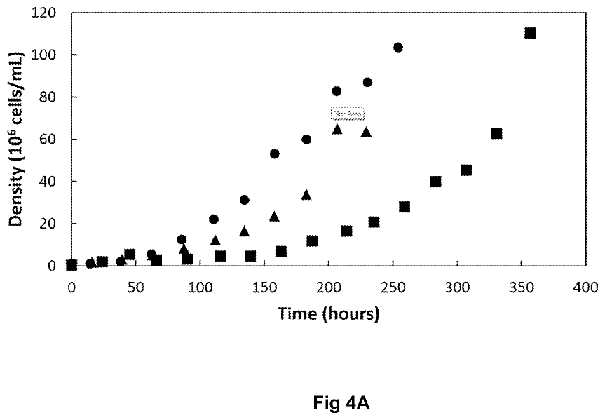

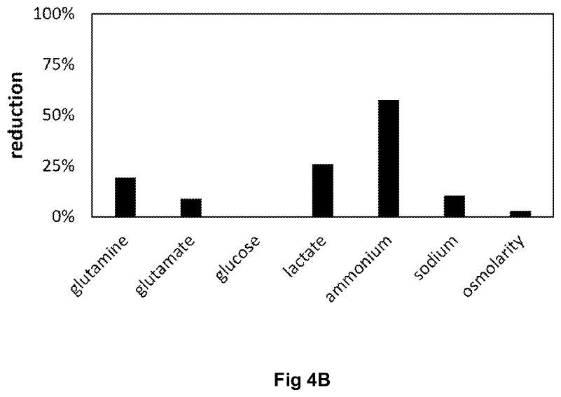

A comparison was made to test cell growth using the three different perfusion systems. The concentrate pH and osmolarity ware adjusted to pH 7 and 300 mOsm/kg, respectively. FIG. 4A presents cell growth over time in a 2 L bioreactor using three different perfusion systems: perfusion with a fresh media, perfusion with a mixture of recycled media and fresh media and perfusion with mixture of rejuvenated media and fresh media. The lag time of the three different curves were between 2 to 4 days. The perfusion used only fresh media reached to 1.1×107 cells/mL after 15 days. The perfusion used a mixture of recycled media and fresh media reached to 6.5×107 cells/mL after 9 days. The cell density at day 10 was similar to the cell density day 9. The perfusion used a mixture of rejuvenated media and fresh media reached to a cell density of 1.03×107 cell/mL after 11 days.

The rejuvenation reduction of glutamine, glutamate, glucose, lactate, ammonium, sodium and osmolarity are given in FIG. 4B. The reduction was found to be 19% for glutamine, 9% for glutamate, 26% for lactate, 58% for ammonium, 10% for sodium and 3% for the osmolarity. The glucose was concentrated by 36% in the rejuvenation phase.

Table 1 compares between the three perfusions run. Using recycled or rejuvenated media saved 35% of fresh media and reduced the maximal perfusion rate from 26 L/day to 10 L/day.

TABLE 1
A comparison between cell growth runs
using three different perfusion feeds.

| | | Recycled | Rejuvenated |
|---|---|---|---|
| | Fresh | media + | media + fresh |
| | media | fresh media | media |
| Cell Total | 110 | 65 | 103 |
| (10⁹ cells) | | | |
| Max Perfusion Rate | 26 | 10 | 10 |
| (L/Day) | | | |
| Fresh Media (L) | 96 | 33 | 35 |
| Recycled Media | — | 29% | 27% |
| Rejuvenation | [symbol] | [symbol] | ✓ |

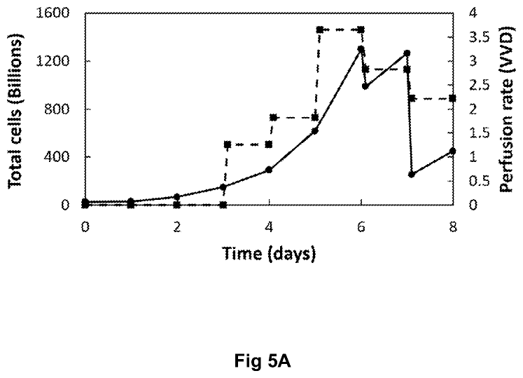

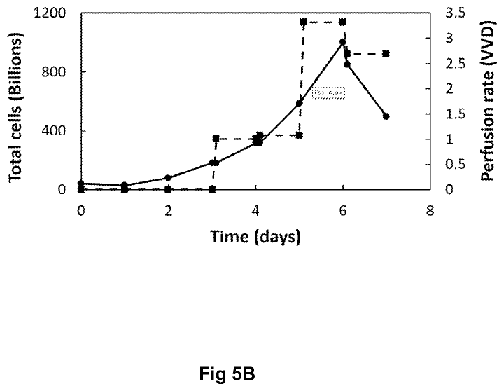

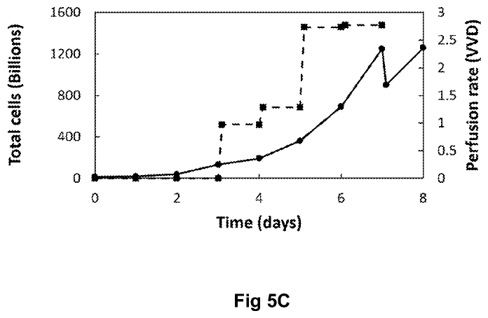

FIG. 5A-C examples of pilot scale production runs of immortalized fibroblasts, SCF-2 cells. Cells were grown in a 270 L BR, using a 50 kDa ATF system and up to 50% rejuvenation at the perfusion phase. The production runs produced between 1.6 to 3.6 kg of biomass solids for laboratory tests from each run. The cell densities in these runs ranged from 1.1×107 to 1.8×107 cells/mL. The biomass produced from the runs, as shown in FIG. 5A-C, were sent for nutritional analysis, and were compared to commercial chicken breast and chicken fat (see Table 2). The moisture content of the biomass was higher than both chicken breast and chicken fat. The protein content was 85% of the chicken breast (73-76 g/100 g and 85 g/100 g, respectively). The sodium amounts ranged from 426 mg/100 g to 636 mg/100 g and were lower by at list 82% than the amounts of the commercial chicken. The cholesterol ranged from 1521 mg/100 g and are an order of magnitude higher than the values found in commercial chicken. The saturated fat values ranged from 6 g/100 g to 8 g/100 g and were higher than values of chicken breast (2 g/100 g) but lower than the values found in chicken fat (21 g/100 g). Dioxins & PCB, antibiotics, pesticides, and melamine were below the detection limits.

TABLE 2
Nutritional analysis of the biomass produced in
the three-pilot scale production runs

| | Chicken | Run | Run | Run | Chicken |
|---|---|---|---|---|---|
| | Breast | I | II | III | Fat |
| Moisture (g/100 g) | 74% | 96% | 96% | 97% | 44% |
| Protein (g/100 g) | 85 | 73 | 73 | 76 | 10 |
| Ash (g/100 g) | 5 | 6 | 5 | 5 | 2 |
| Sodium (mg/100 g) | 759 | 616 | 636 | 426 | 753 |
| Cholesterol | 266 | 1741 | 2344 | 1521 | 144 |
| (mg/100 g) | | | | | |
| Fat by hydrolysis | 7 | 15 | 26 | 20 | 67 |
| (g/100 g) | | | | | |
| Saturated fat (g/100 g) | 2 | 6 | 8 | 7 | 21 |
| Dioxins & PCB | <0.2 | <0.2 | <0.2 | <0.2 | <0.2 |
| Antibiotics | ND | ND | ND | ND | ND |
| Pesticides | ND | ND | ND | ND | ND |
| Melamine | ND | ND | ND | ND | ND |

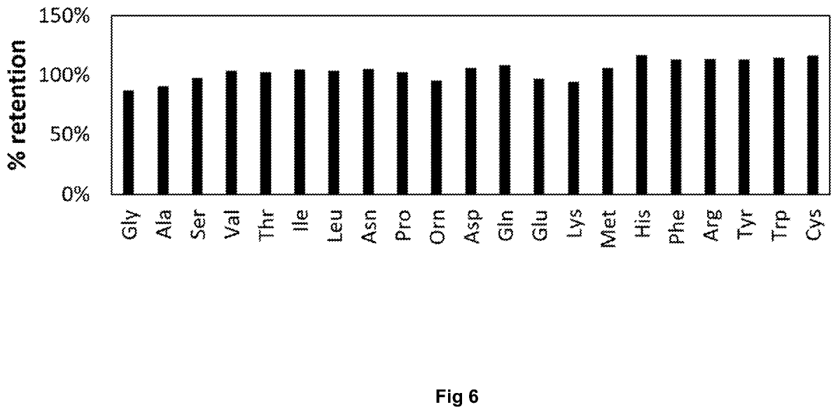

The amino acid retentions of Run I are provided in FIG. 6. Most of the amino acids were enriched by at least 2%. The concentration of alanine, serine, omithine, glutamate and lysine decreased by less than 10% (9%, 2%, 5%, 3% and 6%, respectively). The glycine concentration decreased by 13%.

### Example 3: Selection of Resin Type in Ion Exchange Treatment

Resins Regeneration and Pre-treatment: The ion exchange (IEX) resins used in these studies were regenerated prior the adsorption tests. The resins categorized as cation resin type (strong acid cation) were regenerated with 3-5 bed volumes of 7% HCl (Sigma-Aldrich), using a contact time of 30-45 min. The resins categorized as anion resins (strong or weak base anion) were regenerated with 3-5 bed volumes of 4% NaOH (Sigma-Aldrich), using a contact time of 30-45 min. The regeneration process was followed by a rinsing step with an excess of deionized water until the effluents osmolarity was less than 3 mOsm/kg, and the pH was neutral.

Resin Bed Screening: Various types of resin and their combinations were used to study the adsorption of lactate, ammonium and sodium. The screening was performed using a DMEM medium (DMEM—high glucose, Sigma-Aldrich) spiked with sodium lactate (Sigma-Aldrich), ammonium chloride (Sigma-Aldrich) and sodium chloride (Sigma-Aldrich). Two types of IEX experiments were performed. A screening test was carried in a multi-well plates. The reduction of lactate, ammonium and osmolarity was tested after reaching equilibrium (after 30 min). The plate was stirred at 120 rpm. In addition, the reduction of lactate, ammonium and osmolarity were also tested in packed columns. The lactate, ammonium and osmolarity levels were measured by Accutrend Plus (Roche), Flex 2 (Nova Biomedical) and Fiske Micro-Osmometer Model 210, respectively.

The strong/weak base anion types of resins were aimed at reducing the lactate levels, while the strong acid cation type was aimed at reducing the osmolarity and the ammonium levels. In addition, several combinations of mixed bed were also tested, some of which were mixtures of strong/weak base anion type and strong acid cation type (55:45% wt), while others were a commercialized mixed bed (MB400, Zalion, MR300 and MB20).

Two types of cation resin were tested without combining these reins with anion resin type (FPC88 and IRA210). The lactate adsorption in these cases was zero. The lactate adsorption was also tested using only anion resin type (FPA55) and showed relatively low reduction (10.5%). Combining cation and anion resin types as mixed bed showed more than 30% for combination of FPC88 with IRA67, IRA400 and IRA400 showed lactate adsorption of more than 30% (31.6%, 31.0% and 37.7%, respectively); and FPA55 combined with Dowex C, HPR1200 and Amberlyst 36 (31.7%, 30.0% and 30.0%, respectively). Lactate adsorption of more than 20% were given for mixed bed of FPC88 with FPA55, Lweatit 64, AmberJet 4200, Lewatit MP-62, Lewatit 1065, IRA-410 and HPR4800 (26.8%, 21.6%, 20.5%, 22.9%, 26.2%, 22.5%, and 25.0% respectively); mixed bed of FPA55 with Dowex MSC (26.7%); mixed bed of FPC23 with HPR4800 and IR210 (26.7% and 20.6%, respectively); and mixed bed of MB400, Zalion and MR300 (20.9%, 23.9% and 22.1%, respectively).

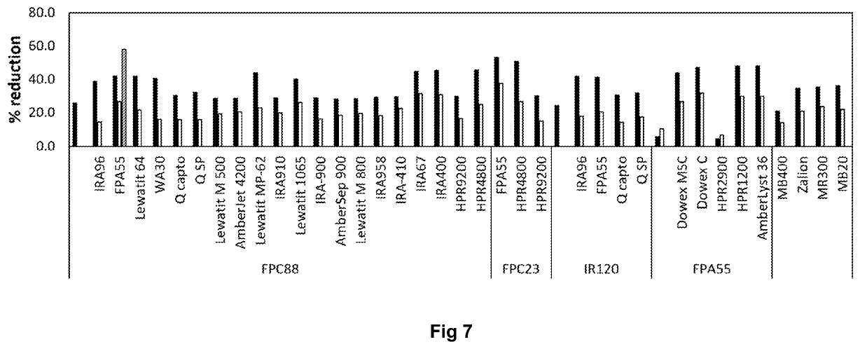

FIG. 7 shows osmolarity reduction of 25.8% for FPC88 bed. When this type of resin was mixed with anion resin type the osmolarity reduction was more than 40% for mixtures of FPC88 with FPA55, Lewatit 64, WA30, Lewatit MP-62, Lewatit 1065, IRA67, IRA400 and HPR4800 (42.0%, 41.8%, 40.6%, 44.0%, 40.4%, 45.0%, 45.6% and 45.9%, respectively). The osmolarity reduction of IR120 was 24.3%. When this resin was mixed with IR96 and FPA55, the osmolarity reduction was more than 40% (41.8% and 41.4, respectively). The mixed beds of MB400, Zalion, MR300 and MB20 showed osmolarity reduction of 21.0%, 34.7%, 35.4% and 36.4%, respectively. The osmolarity was reduced to more than 50% for mixed beds of FPC23 with FPA55 and HPR4800 (53.2% and 50.9%, respectively). The ammonium reduction was tested only for mixed bed of FPC88 and FPA55 and showed reduction of 58.1%.

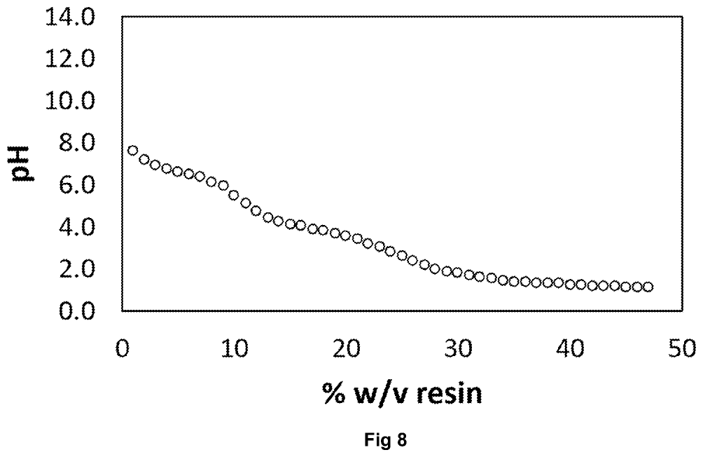

FIG. 8 shows the equilibrium pH using a resin bed of AmberLite FPC88 in DMEM medium spiked with sodium lactate and sodium chloride. In the absence of resin, the pH was 7.6, and was reduced gradually when resin mass was added. The pH reached saturation at 7.5% wt resin concentration to a value of pH 1.2.

### Example 4: Effect of Membrane Type and DH on Nanofiltration

The nanofiltration (NF) in these studies was carried out using several spiral wound membranes (Table 1), having an active area of 2.3-2.6 m². The filtrated medium was sometimes acidified prior the NF treatment, either by adding HCl (Sigma-Aldrich) or by packed cation exchange column. The nanofiltration was also performed as diafiltration mode. In the diafiltration, the medium was pre-diluted with deionized water before the NF stage. The samples taken from the NF feed, concentrate and permeate were analyzed by Flex 2 (Nova Biomedical).

TABLE 3
Nanofiltration membrane

| Membrane | Manufacturer |
|---|---|
| DK | Suez |
| NF-270 | DuPont |
| NFX | Synder |
| TS40 | TriSep |
| DL | Suez |
| MPS | Koch |
| NFS | Synder |

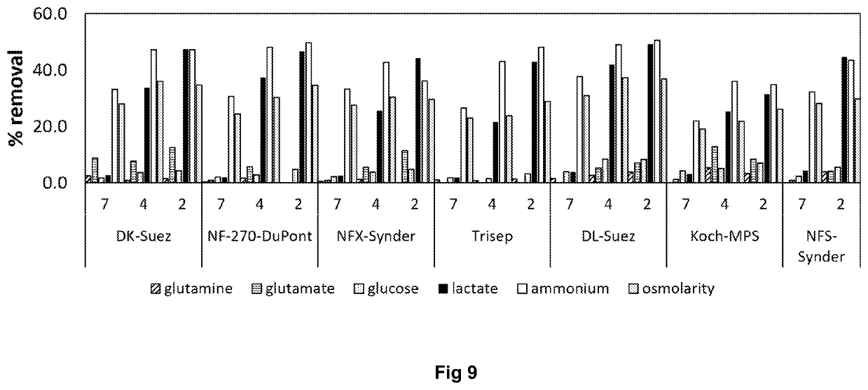

Several types of nanofiltration (NF) membranes (Table 3) were tested for lactate, ammonium and osmolarity reduction in pH 2, 4 and 7 (FIG. 9). In addition, the glucose, glutamine and glutamate concentrations were also measured to estimate the selectivity of these membranes. FIG. 9 shows that the lactate removal was enhanced when the pH was low. For example, the lactate removal using DL membrane was 3.8% at pH 7, while for pH 4 it was 41.9% and 49.2% at pH 2. The lactate removal using TS40 membrane was only 1.9% at pH 7, while for pH 4 it was 21.5% and 42.9% for pH 2.

FIG. 9 shows that the minimal ammonium reduction was given for pH 7 for all screened membranes. The screening showed similar removal of ammonium at pH 2 and 4 for DK (47.2-47.3%), NF-270 (48.1-49.6%), DL (48.9-50.6%) and MPS (36.0-34.9%). A local optimum of ammonium removal was given for NFX (42.8%). For TS40 membrane the ammonium removal at pH 2 (48.1%) was higher than that of pH 4 (43.1%). The NF screening test showed that the lowest osmolarity removal was at pH 7 for all screened NF membranes. The osmolarity reduction at pH 2 and at pH 4 was similar for DK, NFX, DL and MPS at pH 2 and pH 4 was similar (34.7-35.9%, 29.5-30.4% and 36.8-27.2%, respectively). The osmolarity reduction of NF270, TS40 and MPS was maximal at pH 2 (34.5%, 28.8% and 26.0%, respectively). The glutamine removal was less than 5.4% for all screened membranes. The glutamate removal over DK was the highest among all screened membranes for all tested pH values (8.8%, 7.7% and 12.5% for pH 2, 4 and 7, respectively). A significant removal of glutamate was also seen in NFX at pH 2 (11.4%) and MPS at pH 4 (12.8%). No significant trend over the pH was observed for both of these amino acids. The glucose removal was less than 5.4%, expect of the cases of DL at pH 4 and 2 (8.4% and 8.2%, respectively) and MPS at pH 2 (14%).

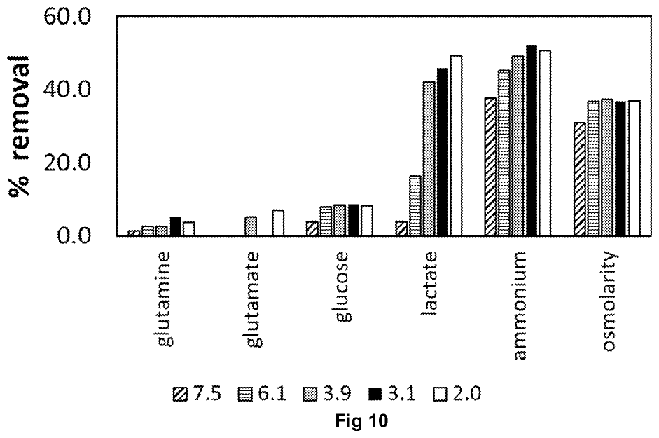

The pH effect on the NF separation was tested on DL membrane (FIG. 10). The lactate removal was increased with reducing the pH, while the main effect was observed between pH 6.1 to 3.9 (30.8%, 160.3%, 41.9%, 45.8% and 49.2% for pH 7.5, 6.1, 3.9, 3.1 and 2.0, respectively). The ammonium removal showed local optimum at pH 3.1 (37.7%, 45.1%, 48.9%, 52.2% and 50.6% for pH 7.5, 6.1, 3.9, 3.1 and 2.0, respectively). Minimal osmolarity reduction was given at pH 7.5 (31.0%), and was similar for more acidic pH (36.7-37.2%). Minimal glucose removal was given at pH 7.5 (3.9%), and was similar for lower pH (8.0%-8.6%). The glutamine removal was relatively low (up to 5.2%) in comparison to lactate, ammonium and osmolarity. The glutamine removal showed a local maximum at pH 3.1 (5.2%). The glutamate removal was up to 7.1%, and no clear trend was observed over the pH range.

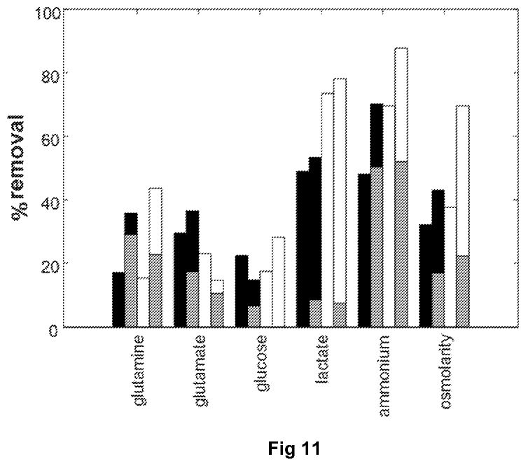

The acidification mechanism effect on the nanofiltration performance was tested (FIG. 11). The lactate removal at the NF stage using either IEX or HCl pre-treatments where similar (44.9% and 49.0% for NF pre-treated by IEX and HCl, respectively; and 70.6% and 73.5% for diafiltration pre-treated by IEX and HCl, respectively). The diafiltration improved the total lactate reduction from 49.0% for NF to 73.5% for diafiltration using HCl pre-treatment, and from total reduction of 53.3% using IEX and NF to 78.1% using IEX and diafiltration. The reduction of the ammonium at the cation exchange column was similar to the reduction when using NF with HCl pre-treatment (52.0-50.4% and 48.1%, respectively). The total ammonium reduction was improved by performing diafiltration from 48.1% for NF to 70.2% for diafiltration using HCl pre-treatment, and from 69.6% to 87.8% when the pre-treatment was IEX. The osmolarity reduction was similar for NF and diafiltration when the pre-treatment was HCl. The osmolarity reduction was improved from 26.2% for NF to 47.3% using diafiltration. The glucose was nearly removed at the IEX treatment (up to 6.5%). However, the total reduction of glucose was up to 28.2%. The glutamine and glutamate removals where 10.5-29.1% at the IEX treatment, and up to 43.6% total removal (maximal removal was observed at the diafiltration process pre-treated by IEX).

### Example 5: Effect of Ultrafiltration

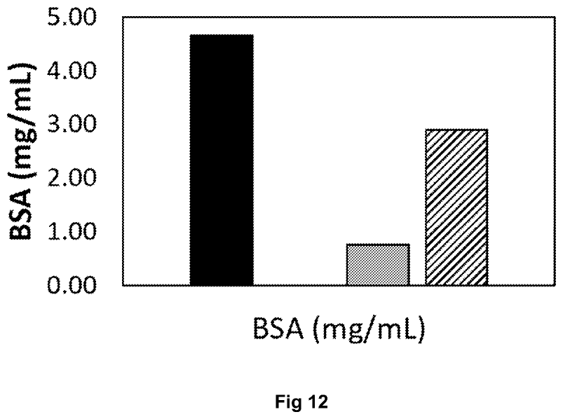

The effect of ultrafiltration was tested as follows: the initial media was harvested from a bioreactor and the ultrafiltration was performed with a UF10 (TriSep™) membrane. The UF was operated by recirculating the media through the membrane. The effect of the UF prior the NF stage was tested by flow test through a 0.22 μm PVDF filter (8.5 cm²). The media was fed through the microfilter under constant pressure of 1.5 bar. All the samples were kept at 37° C. prior this assay. The nanofiltration was carried out after reducing the pH to 2.8 by hydrochloric acid addition, with a DK membrane. The concentrate stream of each treatment (with and without prior step of ultrafiltration) was neutralized to pH 7.1 and diluted to 300 mOsm/kg. The BSA concentration was measured at 280 nm (NanoDrop One, Thermo Scientific). FIG. 12 represents the protein content at the feed of the rejuvenation stage and the rejuvenated media with and without prior UF stage. The UF reduced the BSA concentration from 4.7 mg/mL to below the limit of detection. After concentrating this media at the NF stage, the BSA concentration was 0.8 mg/mL (83% reduction). This process was repeated without UF stage, and the BSA concentration was 2.9 mg/mL (38% reduction).

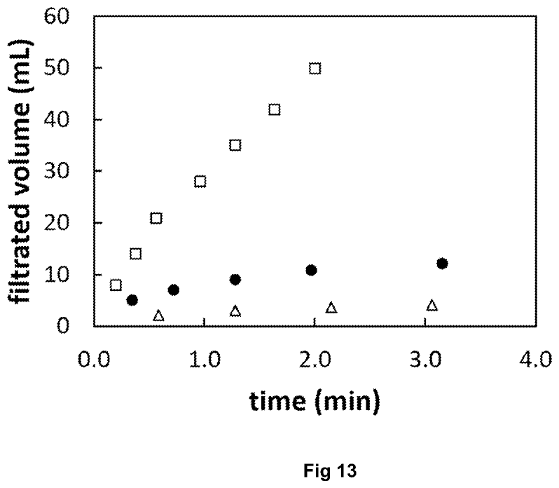

The effect of the UF prior step on the flowrate through a microfilter was tested (FIG. 13). The flowrate of the rejuvenated media that was pre-filtrated with UF was nearly constant (R2=0.9908). The average flow was 22.5 mL/min. The average flowrate of the rejuvenated media that was pre-treated with UF was 2.4 mL/min. The flowrate of this sample decreased from 14.3 mL/min after 0.4 min to 0.8 mL/min after 3.2 min. The average flowrate of the rejuvenated media that was not filtrated with UF was 0.8 mL/min. The flowrate in this case decreased from 3.4 mL/min after 0.6 min to 0.5 mL/min after 3.1 min.

### Discussion for Examples 2-5

A growth medium might contain a toxic level of several waste materials such as lactate and ammonium. The production of lactic acid in cells also indirectly causes osmolarity to increase due to the action of the pH control circuit. IEX treatment is capable of reducing these toxic effects by adsorption of ammonium and sodium on cation resin, and by adsorption of lactate and chlorides on anion resin. However, this treatment is not selective to these waste materials only. It might also absorb growth factor such as amino acids or vitamins.

The screening test showed a few promising anion type resins for lactate removal: IRA410, IRA67, HPR4800 and FPA55. The osmolarity removal screening test showed that mixing FPC88 with anion type of resin always achieved better osmolarity reduction when mixing this type of resin than using the FPC88 only. These results may point a synergistic effect when using two different types of resins (anion and cation). The cation resin, which were pre-loaded with protons, reduced the pH due to exchange of cation in the medium with these protons. On the other side, the anion resin, which was pre-loaded with hydroxyl, increased the pH due to exchange of anions in the medium with the pre-loaded hydroxyls. The dynamics of the adsorption was probably related to the pH dynamics. When using a bed comprised of only FPC88 the pH was reduced very fast (strong acid active group, sulfonic acid), and reached to early equilibrium, i.e., the limiting factor was the pH value. On the other hand, the combination with anion resin type, which added hydroxyls to the medium, postponed the equilibrium and allowed exchanging of cations for longer residence time; therefore, more cations were adsorbed on the resin. Hence, the limiting factor was shifted to the number of active sites of the resin.

Glutamine and glutamate are examples for two amino acids that has similar size (146 Da) but are differed in their molecular charge (glutamate is less probable to be find positive than glutamine in pH 2). The reduction of glutamine was higher than the reduction of glutamate on a rejuvenation process based on cation exchanging and nanofiltration. The difference between glutamine and glutamate retention therefore is not related to their molecular size, but to their molecular charge. The cation exchanging using H-charged resin, reduces the pH due to exchanging of cation molecules by hydrogen to the medium. Consequently, some molecules change their molecular charge, and their affinity to the cation exchange bed will affected. Since glutamine is more likely to be find positive in pH 2, its affinity to the FPC88 was higher.

The nanofiltration membranes, differed from each other mostly by their MWCO and their active layer polymer, were used in this study to remove waste materials such as lactate, ammonium and osmolarity, while also aimed at retaining growth factors such as amino acids. The nanofiltration performance were affected mostly by the membrane type and the pH set-point. All screened members showed satisfying lactate removal (at list 44.7% at pH 2). NFX and MPS were limited in their ammonium and osmolarity reductions (less than 37% for ammonium reduction, whereas at list 43.5% for other membranes; less than 30% osmolarity reduction, whereas at list 34.5% for other membranes). TS40 and NFS were limited only by osmolarity reduction.

Lactic acid, ammonia and amino-acids dissociation degree is pH dependent. For example, in case of pH reduction below the pKa of lactate (3.8) (Ecker et al., 2012, Journal of Membrane Science, 389: 389-398), the presence of the neutrally charged lactic acids is higher than the presence of the negatively charged lactate ion. While for pH above 3.8, more lactate ions are presence than lactic acid. The pH also affects the membrane effective pore size. At a pH value that is higher than the isoelectric point of the membrane (e.g., 4.8 for DL and 4.0 for DK) (Chandrapala et al., 2016, Separation and Purification Technology, 160: 18-27), the carboxylic groups at the membrane becomes negatively charged. Consequently, there is more electrostatic repulsion between the carboxylic groups and the effective pore size is increased. At a pH that is lower than the isoelectric point of the membrane, the carboxylic groups become uncharged; hence, the effective pore size is decreased. These two pH effects change the membrane separation due to charge interactions between the dissolved ions and the membrane side groups and change in the effective pore size. The result is more pronounced lactate removal at more acidic pH values. The selectivity to glucose is increased when reducing the pH due to the pore size effect.

Another critical parameter that determines the molecules retention in a nanofiltration process is the molecular size. Glycine and alanine are the two smallest amino acids (75 and 89 Da, respectively). The retention of these two amino acids was the lowest among all other amino acids (87% and 91, respectively).

The selectivity of the rejuvenation process was found to be related to the pH conditions. The pH range which was found to be optimal might cause to a denaturation of proteins that present at the medium. The denaturation disrupts the spatial arrangement of the proteins and their non-covalent interactions. Hence, the activity of the protein and their solubility might change. To avoid these negative effects, separation of proteins is needed. The protein separation can be performed by ultrafiltration (UF). The protein content at the rejuvenated media that was pre-filtrated with UF was lower by 72% in comparison to rejuvenated media that was not filtrated with UF. Moreover, the protein separation phase eliminated clogging of the microfilter, which is sometimes needed for aseptic conditions.

At high cell densities, the cell growth can be limited by the lack of nutrients or by the presence of produced metabolites that have inhibitory effect. Lactate, for example, inhibits cell growth. Moreover, to overcome the pH reduction of the media due to the production of lactic acid from the cells, an alkaline solution is usually added as a part of a pH control circuit of the bioreactor; hence, the osmolarity, which is an additional limiting factor, increases. Therefore, at high cell densities, continuous supplement of nutrients and reduction of inhibitors is a critical strategy to maintain the log phase of the cells. Feeding fresh media in a perfusion process can supply nutrients and dilute the inhibitors concentration at the bioreactor, but this might require large amounts of fresh media and to be too expensive for a food tech process. Recycling the media at a perfusion process can supply nutrients that were not fully consumed by the cells, and to reduce the required volume of fresh media by some extent. However, inhibitors will be recycled to the bioreactor as well. Media rejuvenation can be optimized to selectively remove these inhibitory metabolites, while retaining essential nutrients in the media.

The perfusion mode was tested using three different feeds for a 2 L bioreactor perfusion process. Recycling the media reduced the required amounts of fresh media however, the maximal cell density was only 59% of the perfusion that used fresh media. A possible reason of entering to the stationary phase earlier is the presence of inhibitors such lactate, ammonium and osmolarity. Recycling similar ratio of rejuvenated media allowed the cell growth to reach to similar cell densities to fresh feed perfusion. The rejuvenation treatment decreased the lactate and ammonium by 26% and 58% and enabled continuous cell growth.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A system for recycling a cell culture medium, the system comprising:
a) means for removing a cell culture medium from a bioreactor;
b) means for filtering the cell culture medium, thereby obtaining a waste medium and a concentrate medium, wherein the waste medium comprises at least one waste material and is essentially devoid of cells and large proteins and is further processed, wherein the concentrate medium is circulated back into the bioreactor;
c) means for acidifying the waste medium; and
d) means for subjecting the acidified waste medium to nanofiltration, thereby removing the at least one waste material from the waste medium and obtaining a rejuvenated medium that is diminished or essentially devoid of the at least one waste material, wherein the rejuvenated medium is further processed and circulated back into the bioreactor, thereby recycling the cell culture medium.

2. The system of claim 1, wherein the cell culture medium comprises one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials.

3. The system of claim 2, wherein the cell culture medium comprises blood cells.

4. The system of claim 2 or 3, wherein the at least one waste material interferes with desired growth and/or desired differentiation of the cells.

5. The system of claim 2, wherein the cell culture medium comprises tissues cultured for antibody production or cultured meat production.

6. The system of any preceding claim, wherein the filtering means comprises at least one hollow fiber with a pore cutoff of up to 60 kDa.

7. The system of claim 6, wherein the at least one hollow fiber has a pore density of at least 10% of the inner wall surface of the hollow fiber.

8. The system of any one of claims 1 to 5, wherein the filtering means comprises continuous centrifugation.

9. The system of claim 8, wherein the centrifuge operates at 1000 to 20,000×g, preferably at 8400×g, thereby removing cells and large proteins from the waste medium.

10. The system of claim 8 or 9, wherein the centrifugation continuously removes cell mass from the bioreactor, thereby maintaining a constant cell density over the recycling period.

11. The system of any preceding claim, wherein the at least one waste material has a molecular weight of no greater than 60 kDa.

12. The system of claim 11, wherein the at least one waste material comprises ammonia, lactate, toxins, sodium salts, alanine, glutamic acid, aspartic acid, ammonium, reactive oxygen and nitrogen species, or a combination thereof.

13. The system of claim 12, wherein the at least one waste material comprises ammonia, ammonium, and/or lactate.

14. The system of claim 1, wherein the concentrate medium comprises cells and essential materials for cell growth and/or differentiation.

15. The system of claim 1, wherein the means for acidifying the waste medium comprises subjecting the waste medium to a cation exchange column and/or adding an acid to the waste medium.

16. The system of claim 15, wherein the cation exchange column comprises at least one cation resin.

17. The system of claim 16, wherein the cation exchange column comprises AmberLite FPC88.

18. The system of claim 15, wherein the acid is selected from the group consisting of HCl, sulfuric acid, nitric acid, phosphoric acid, carbonic acid, citric acid, and acetic acid.

19. The system of any one of claims 15 to 18, wherein the acidified waste medium has a pH value of less than 4.

20. The system of claim 19, wherein the acidified waste medium has a pH value of about 2.

21. The system of claim 1, wherein the nanofiltration is also performed as a diafiltration mode which involves pre-diluting the acidified waste medium with deionized water before the nanofiltration.

22. The system of claim 21, wherein the nanofiltration has a molecular weight cutoff of from about 150 to about 300 Da.

23. The system of claim 22, wherein the waste materials are recovered from the acidified waste medium post nanofiltration, wherein the waste materials comprise ammonia, ammonium, and/or lactate, wherein the components are isolated and recovered individually.

24. The system of claim 1, wherein the rejuvenated medium comprises glucose and fatty acids having a molecular weight greater than 150 Da.

25. The system of claim 1, wherein the rejuvenated medium is further processed by means of neutralizing the pH thereof in step (d).

26. The system of claim 25, wherein the neutralizing means comprises subjecting the rejuvenated medium to an anion exchange column.

27. The system of claim 26, wherein the anion exchange column comprises at least one anion resin.

28. The system of claim 27, wherein the anion exchange column comprises FPA55.

29. The system of claim 25, wherein the neutralizing means comprises adding a base to the rejuvenated medium.

30. The system of claim 29, wherein the base is selected from the group consisting of NaOH, sodium bicarbonate, potassium hydroxide, magnesium hydroxide, and calcium hydroxide.

31. The system of any one of claims 25 to 30, wherein the pH of the rejuvenated medium is adjusted to pH>6.

32. The system of claim 31, wherein the rejuvenated medium has a pH of about 7.

33. The system of any one of claims 25 to 32, wherein the osmolarity of the rejuvenated medium is adjusted to be less than 360 milliosmoles per kilogram (mOsm/kg) of water.

34. The system of claim 33, wherein the rejuvenated medium has an osmolarity of about 280 mOsm/kg.

35. The system of any preceding claim, wherein biomass is expanded in the cell culture medium to produce cultured meat.

36. The system of claim 1, optionally comprising an ultrafiltration means before (c).

37. A method for recycling a cell culture medium, the method comprising:
a) removing a cell culture medium from a bioreactor;
b) filtering the cell culture medium, thereby obtaining a waste medium for further processing and a concentrate medium for recirculation, wherein the waste medium comprises at least one waste material and is essentially devoid of cells and large proteins;
c) acidifying the waste medium; and
d) subjecting the acidified waste medium to nanofiltration, thereby removing the at least one waste material therefrom and obtaining a rejuvenated medium that is diminished or essentially devoid of the at least one waste material for recirculation, thereby recycling the cell culture medium.

38. The method of claim 37, wherein the cell culture medium comprising one or more materials selected from the group consisting of cells, tissues, nutrients, supplements, feeds, amino acids, peptides, proteins, vitamins, polyamines, sugars, carbohydrates, lipids, nucleic acids, hormones, fatty acids, trace materials and waste materials.

39. The method of claim 38, wherein the cell culture medium comprises blood cells.

40. The method of claim 38 or 39, wherein the waste materials interfere with desired growth and/or desired differentiation of the cells.

41. The method of claim 38, wherein the cell culture medium comprises tissues cultured for antibody production or cultured meat production.

42. The method of any one of claims 37 to 41, wherein the cell culture medium is filtered through at least one hollow fiber with a pore cutoff of up to 60 kDa in step (b).

43. The method of claim 42, wherein the at least one hollow fiber has a pore density of at least 10% of the inner wall surface of the hollow fiber.

44. The method of any one of claims 37 to 41, wherein the cell culture medium is filtered through continuous centrifugation.

45. The method of claim 44, wherein the centrifuge operates at 1000 to 20,000×g, preferably at 8400×g, thereby removing cells and large proteins from the waste medium.

46. The method of claim 44 or 45, wherein the centrifugation continuously removes cell mass from the bioreactor, thereby maintaining a constant cell density over the recycling period.

47. The method of any one of claims 37 to 46, wherein the at least one waste material has a molecular weight of no greater than 60 kDa.

48. The method of claim 47, wherein the at least one waste material comprises ammonia, lactate, toxins, sodium salts, alanine, glutamic acid, aspartic acid, ammonium, reactive oxygen and nitrogen species, or a combination thereof.

49. The method of claim 48, wherein the at least one waste material comprises ammonia, ammonium, and/or lactate.

50. The method of claim 37, wherein the concentrate medium comprises cells and essential materials for cell growth and/or differentiation.

51. The method of claim 37, wherein the waste medium is subjected to a cation exchange column and/or addition of an acid in step (3).

52. The method of claim 51, wherein the cation exchange column comprises at least one cation resin.

53. The method of claim 52, wherein the cation exchange column comprises AmberLite FPC88.

54. The method of claim 51, wherein the acid is selected from the group consisting of HCl, sulfuric acid, nitric acid, phosphoric acid, carbonic acid, citric acid, and acetic acid.

55. The method of any one of claims 51 to 54, wherein the acidified waste medium has a pH value of less than 4.

56. The method of claim 55, wherein the acidified waste medium has a pH value of about 2.

57. The method of claim 37, wherein the nanofiltration is also performed as a diafiltration mode which involves pre-diluting the acidified waste medium with deionized water before the nanofiltration.

58. The method of claim 57, wherein the nanofiltration has a molecular weight cutoff of from about 150 to about 300 Da.

59. The method of claim 58, further comprising:
a) recovering the waste materials from the acidified waste medium post nanofiltration, wherein the waste materials comprise ammonia, ammonium, and/or lactate;
b) isolating the components of the waste materials; and
c) recovering the individual component.

60. The method of claim 37, wherein the rejuvenated medium comprises fatty acids having a molecular weight greater than 150 Da and glucose.

61. The method of claim 37, wherein the pH of the rejuvenated medium is further neutralized in step (4).

62. The method of claim 61, wherein the rejuvenated medium is subjected to an anion exchange column.

63. The method of claim 62, wherein the anion exchange column comprises at least one anion resin.

64. The method of claim 63, wherein the anion exchange column comprises FPA55.

65. The method of claim 61, wherein a base is added to the rejuvenated medium for neutralization.

66. The method of claim 65, wherein the base is selected from the group consisting of NaOH, sodium bicarbonate, potassium hydroxide, magnesium hydroxide, and calcium hydroxide.

67. The method of any one of claims 61 to 66, wherein the pH of the rejuvenated medium is adjusted to pH>6.

68. The method of claim 67, wherein the rejuvenated medium has a pH of about 7.

69. The method of any one of claims 61 to 68, wherein the osmolarity of the rejuvenated medium is adjusted to be less than 360 milliosmoles per kilogram (mOsm/kg) of water.

70. The method of claim 69, wherein the rejuvenated medium has an osmolarity of about 280 mOsm/kg.

71. The method of any one of claims 40 to 70, wherein the cell culture medium is used to grow cultured meat.

72. The method of claim 37, further comprising an ultrafiltration step prior to step c.

73. A method for expanding cells in a bioreactor, said method comprising:
a) culturing cells in a cell culture medium comprising nutrients and waste molecules; and
b) recycling the cell culture medium according to the method of any one of claims 36 to 69 to reduce the amount of waste molecules or remove the waste molecules from the medium.

74. The method of claim 73, wherein the expanded cells are used to produce cultured meat.

75. A method for reducing or removing waste products from a patient's blood, said method comprising:
a) obtaining blood from the patient using dialysis;
b) filtering blood to obtain protein-free plasma containing waste products;
c) recycling the protein-free plasma according to the method of any one of claims 36 to 67 to reduce the amount of waste products or remove the waste products from the plasma.
